(19) United States
(12) Patent Application Publication
(10) Pub. No.: US 2005/0124620 A1
(43) Pub. Date: Jun. 9, 2005

(54) Pharmaceutical compounds

(76) Inventors: Martyn Frederickson, Cambridge (GB); Adrian Liam Gill, Cambridge (GB); Alessandro Padova, Roma (IT); Miles Stuart Congreve, Cambridge (GB)

Correspondence Address: HESLIN ROTHENBERG FARLEY & MESITI PC, 5 COLUMBIA CIRCLE, ALBANY, NY, 12203, US

(21) Appl. No.: 10/962,085

(22) Filed: Oct. 8, 2004

Related U.S. Application Data
(63) Continuation of application No. PCT/GB03/01507, filed on Apr. 8, 2003.

(30) Foreign Application Priority Data
Apr. 9, 2002 (GB) 0208248.5
Jun. 29, 2002 (GB) 0215180.1

(51) Int. Cl. C07D 471/02; C07D 487/02; A61K 31/53; A61K 31/519

(52) U.S. Cl. 514/243; 514/248; 514/249; 514/265.1; 544/183; 544/280; 544/300; 546/113; 514/300

(57) ABSTRACT

Compounds are disclosed of the formula (I): 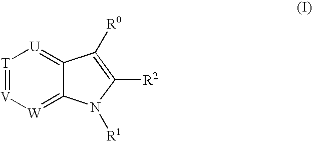
in which U, T, V and W are each a nitrogen atom or carbon atom. When U, T, V or W is a carbon atom, it may be substituted. The compounds are inhibitors of p38 MAP kinase and are useful for treating inflammatory diseases such as arthritis. An example of such a compound is:

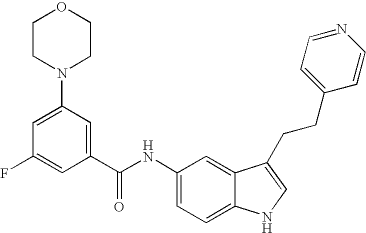

This invention relates to compounds that inhibit or modulate the activity of p38 MAP kinase, to the use of the compounds in the treatment or prophylaxis of disease states or conditions mediated by p38 MAP kinase, and to novel compounds having p38 MAP kinase inhibitory or modulating activity. Also provided are pharmaceutical compositions containing the compounds and novel chemical intermediates.

### BACKGROUND OF THE INVENTION

Protein kinases constitute a large family of structurally related enzymes that are responsible for the control of a wide variety of signal transduction processes within the cell (Hardie, G. and Hanks, S. (1995) The Protein Kinase Facts Book. I and II, Academic Press, San Diego, Calif.). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks, S. K., Hunter, T., FASEB J., 9:576-596 (1995); Knighton, et al., Science, 253:407-414 (1991); Hiles, et a., Cell, 70:419-429 (1992); Kunz, et al., Cell, 73:585-596(1993); Garcia-Bustos, etal., EMBO J., 13:2352-2361 (1994)).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signaling processes, by adding phosphate groups to target proteins. These phosphorylation events act as molecular on/off switches that can modulate or regulate the target protein biological function. Phosphorylation of target proteins occurs in response to a variety of extraceliular signals (horrnones, neurotransmitters, growth and differentiation factors, etc.), cell cycle events, environmental or nutritional stresses, etc. The appropriate protein kinase functions in signaling pathways to activate or inactivate (either directly or indirectly), for example, a metabolic enzyme, regulatory protein, receptor, cytoskeletal protein, ion channel or pump, or transcription factor. Uncontrolled signaling due to defective control of protein phosphorylation has been implicated in a number of diseases, including, for example, inflammation, cancer, allergy/asthma, disease and conditions of the immune system, disease and conditions of the central nervous system, and angiogenesis.

The mitogen-activated protein (MAP) kinase family consists of a series of structurally related proline-directed serine/threonine kinases that are activated either by growth factors (such as EGF) and phorbol esters (ERK), or by IL-1, TNF or stress (p38, JNK). These kinases mediate the effects of numerous extracellular stimuli on a wide array of biological processes, such as cell proliferation, differentiation and death. Three groups of manimalian MAP kinases have been studied in detail: the extraceflular signal-egulated kinases (ERK), the c-Jun NH₂-terminal kinases (JNK) and the p38 MAP kinases.

There are five known human isoforms of p38 MAP kinase, p38α, p38β, p38β2, p38γ and p38δ. The p38 kinases, which are also known as cytokine suppressive anti-inflammatory drug binding proteins (CSBP), stress activated protein kinases (SAPK) and RK, are responsible for phosphorylating (Stein et al., Ann. Rep. Med Chem., 31, 289-298 (1996)) and activating transcription factors (such as ATF-2, MAX, CHOP and C/ERPb) as well as other kinases (such as MAPKAP-K2/3 or MK2/3), and are themselves activated by physical and chemical stress (e.g. UV, osmotic stress), pro-inflammatory cytokines and bacterial lipopolysaccharide (LPS) (Herlaar, E & Brown, Z., Molecular Medicine Today, 5: 439-447 (1999)). The products of p38 phosphorylation have been shown to mediate the production of inflammatory cytokines, including TNF and IL-1, and cyclooxygenase-2 (COX-2).

Each of these cytokines has been implicated in numerous disease states and conditions. IL-I1 and TNF are also known to stimulate the production of other proinflammatory cytolines such as IL-6 and IL-8.

Interleukin-1 (IL-1) and Tumor Necrosis Factor (TNF) are biological substances produced by a variety of cells, such as monocytes or macrophages. IL-1 has been demonstrated to mediate a variety of biological activities thought to be important in immunoregulation and other physiological conditions such as inflammation (e.g. Dinarello, et al., Rev. Infect. Disease, 6:51 (1984)). The myriad of known biological activities of IL-1 include the activation of T helper cells, induction of fever, stimulation of prostaglandin or collagenase production, neutrophil chemotaxis, induction of acute phase proteins and the suppression of plasma iron levels.

There are many disease states in which excessive or unregulated IL-1 production is implicated in exacerbating and/or causing the disease. These include rheumatoid arthritis (Arend etal., Arthritis & Rheumatism 38(2): 151-160, osteoarthritis, endotoxemia and/or toxic shock syndrome, other acute or chronic inflammatory disease states such as the inflammatory reaction induced by endotoxin or inflammatory bowel disease; tuberculosis, atherosclerosis, Hodgkin's disease (Benharroch et al., Euro. Cytokine Network 7(1): 51-57), muscle degeneration, cachexia, psoriatic arthritis, Reiter's syndrome, gout, traumatic arthritis, rubella arthritis, acute synovitis and Alzheimer's disease. Evidence also links IL-1 activity to diabetes and pancreatic B cells (Dinarello, J. Clinical Immunology, 5: 287-297 (1985)). Because inhibition of p38 leads to inhibition of IL-1 production, it is envisaged that p38 inhibitors will be useful in the treatment of the above listed diseases.

Excessive or unregulated TNF production has been implicated in mediating or exacerbating a number of diseases including rheumatoid arthritis (Maini et al, APMIS, 105(4): 257-263), rheumatoid spondylitis, osteoarthritis, gouty arthritis and other arthritic conditions; sepsis, septic shock, endotoxic shock, gram negative sepsis, toxic shock syndrome, adult respiratory distress syndrome, cerebral malaria, chronic pulmonary inflammatory disease, silicosis, pulmonary sarcoisosis, bone resorption diseases, reperfusion injury, graft vs. host reaction, allograft rejections, fever and myalgias due to infection, such as influenza, herpes simplex virus type-1 (HSV-1), HSV-2, cytomegalovirus (CMV), varicella-zoster virus (VZV), Epstein-Barr virus (EBV), human herpes virus-6 (HHV-6), HHV-7, HHV-8, pseudorabies, rhinotracheitis and cachexia secondary to infection or malignancy, cachexia secondary to acquired immune deficiency syndrome (AIDS), AIDS, ARC (AIDS related complex), keloid formation, scar tissue formation, Crohn's disease, ulcerative colitis, or pyresis. Because inhibition of p38 leads to inhibition of TNF production, it is envisaged that p38 inhibitors will be useful in the treatment of the above listed diseases.

Interleukin-8 (IL-8) is a chemotactic factor produced by several cell types including mononuclear cells, fibroblasts, endothelial cells, and keratinocytes. Its production from endothelial cells is induced by IL-1, TNF, or lipopolysachharide (LPS). IL-8 stimulates a number of functions in vitro. It has been shown to have chemoattractant properties for neutrophils, T-lymphocytes, and basophils. In addition it induces histamine release from basophils from both normal and atopic individuals as well as lysozomal enzyme release and respiratory burst from neutrophils. IL-8 has also been shown to increase the surface expression of Mac-1 (CD 11 blCD 18) on neutrophils without de novo protein synthesis; this may contribute to increased adhesion of the neutrophils to vascular endothelial cells. Many diseases are characterized by massive neutrophil infiltration. Conditions associated with an increased in IL-8 production (which is responsible for chemotaxis of neutrophil into the inflammatory site) would benefit from treatment with compounds which are suppressive of IL-8 production. Recently Chronic Obstructive Pulmonary Disease (COPD) has been linked to raised levels of IL-8 (Barnes et al., Curr. Opin. Pharmacol, 1: 242-7 (2001)). Other conditions linked to IL-8 include acute respiratory distress syndrome (ARDS), asthma, pulmonary fibrosis and bacterial pneumonia.

IL-1 and TNF affect a wide variety of cells and tissues and these cytokines as well as other leukocyte derived cytokines are important and critical inflammatory mediators of a wide variety of disease states and conditions. The inhibition of these cytokines is of benefit in controlling, reducing and alleviating many of these disease states.

Inhibition of signal transduction via p38, which in addition to IL-1, TNF and IL-8 described above is also required for the synthesis and/or action of several additional pro-inflammatory proteins (i.e., IL-6, GM-CSF, COX-2, collagenase and stromelysin), is expected to be a highly effective mechanism for regulating the excessive and destructive activation of the immune system. This expectation is supported by the potent and diverse anti-inflammatory activities described for p38 kinase inhibitors (Badger, et al., J. Pharm. Exp. Thera., 279: 1453-1461(1996); Griswold, et al., Pharmacol. Comm., 7: 323-229 (1996)).

WO 01/47922 (Aventis Pharma) discloses a class of substituted azindoles and their use in treating disease states capable of being modulated by inhibition of protein kinases, and in particular the Sykkinase, a 72-kDa cytoplasmic protein tyrosine kinase.

WO 02/10137 (Signal Pharmaceuticals Inc.) discloses a class of indazole compounds as inhibitors of JNK kinases. The compounds are disclosed as having a variety of therapeutic uses such as the treatment of artritis.

WO 01/02369 (Agouron Pharmaceuticals) also relates to indazole compounds that modulate and/or inhibit the activity of certain protein kinases, particularly tyrosine kinases. The compounds have a substituted or unsubstituted aryl or hetero-aryl group in the 3 position of the indazole ring.

WO 00/71535 (Scios Inc.) discloses indole-type compounds as inhibitors of p38 kinase. The 6-membered ring in the indole-like nucleus of the compounds is linked to a piperidine or piperazine group via a short linker group.

WO 00/46198 (Astra Zeneca) discloses a class of indole derivatives having anti-inflammatory activity in which the compounds have an aryl or hetero-aryl ring linked to the 1-position of the indole nucleus by a CH₂ or SO₂ linkcing group. The compounds are disclosed as being antagonists of the pro-inflammatory cytokine MCP-1.

WO 93/1408 (Smith-Kline Beecham) discloses 1,3,4-triaryl imidazoles as inhibitors of p38 MAP kinase.

WO 99/15164 (Zeneca) discloses various bis-benzamidophenyl derivatives compounds which exhibit inhibition of p38 activity.

WO 99/32111 (Bayer) discloses a series of diarylurea compounds which act as p38 MAP kinase inhibitors.

WO 99/00357 (Vertex) discloses a further class of diarylurea compounds as p38 MAP kinase inhibitors.

WO 99/43651 and W099/43654 (both in the name of Genetics Institute) disclose substituted indoles as phospholipase inhibitors useful in treating or preventing inflammatory conditions.

### SUMMARY OF THE INVENTION

The invention provides a class of compounds, some known and some novel, that have p38 MAP kinase inhibiting or modulating activity, and which it is envisaged will be useful in preventing or treating disease states or conditions mediated by the p38 MAP kinases.

Accordingly, in a first aspect, the invention provides a compound for use in the prophylaxis or treatment of a disease state or condition mediated by a p38 MAP kinase; the compound being of the general formula (I): 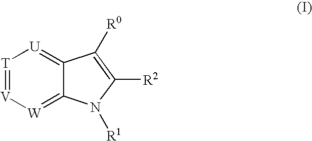
wherein U, T, V and W are each a nitrogen atom or a group CR⁴ provided that no more than three of U, T, V and W are nitrogen atoms;
R⁰ is hydrogen, C₁₋₄ hydrocarbyl, halogen or a group -A-R³;
R¹ is hydrogen, C₁₋₄ hydrocarbyl or a group -A-R³; provided that only one of R⁰ and R¹ is a group -A-R³;
R² is hydrogen, C₁₋₄ hydrocarbyl or halogen;
A is a carbon- or heteroatom-containing linker group having a linking chain length of one or two atoms;
R³ is a monocyclic or bicyclic heteroaryl group containing from five to twelve ring members;
each group R⁴ is independently selected from hydrogen, hydroxy, halogen, nitro, cyano, a monocyclic heterocyclic group having up to seven ring members, a group N(R⁵)₂, a group C(O)N(R⁶)₂, a group SO₂N(R⁶)₂, a group R^{a}—R^{b} and a group Y; provided that no more than one group Y is present;
R^{a} is a bond, O, S, SO, SO₂, NH or N—C₁₋₄ hydrocarbyl;
R^{b} is C₁₋₈ hydrocarbyl optionally interrupted by O, S, SO, SO₂, NH or N—C₁₋₄ hydrocarbyl and optionally substituted by one or more substitutents selected from hydroxy, amino, mono- or di-C₁₋₄ hydrocarbylamino, C₁₋₄ hydrocarbyloxy, oxo, C₁₋₄ hydrocarbylthio and halogen;
each group R⁵ is independently selected from hydrogen, C₁₋₄ alkyl, C₁₋₄ acyl and C₁₋₄ alkylsulphonyl;
each group R⁶ is independently selected from hydrogen and C₁₋₄ hydrocarbyl;
Y is a group —N(R⁷)—C(O)—R⁸ or —N(R⁷)—SO₂—R⁸;
R⁷ is hydrogen, C₁₋₄ hydrocarbyl or a group C(O)—R⁸ or SO₂—R⁸;
R⁸ is selected from C₁₋₁₀ hydrocarbyl, C₁₋₁₀ hydrocarbylamino, C₁₋₁₀ hydrocarbylthio, C₁₋₁₀ hydrocarbyloxy, and aryl, arylamino, arylthio and aryloxy groups, the aryl moieties of which are carbocyclic or heterocyclic and have from five to twelve ring members, each substituent group R⁸ being optionally substituted by one or more groups R⁴ other than Y; or R⁷ and R⁸ together with the nitrogen and carbon or sulphur atoms to which they are attached are linked to form a ring structure of 4 to 7 ring members;
wherein R⁰ is other than a 2-(2,4-diamino-6-triazinyl)ethyl group when, in combination, U, T, V and W are all CH, and R¹ and R² are both hydrogen;
and provided that when the group -A-R³ contains an acidic substitituent group selected from carboxylic, phosphonic and sulphonic acids and tetrazoles, or contains a —C(O)NSO₂— group, or when -A- is —C(O)N— and the nitrogen atom of the group A is linked directly to a furan or thiophene ring, then either R¹ is -A-R³ and both R⁰ and R² are hydrogen, or R⁰ is -A-R³ and R¹ is hydrogen.

Compounds of the formula (1) as defined above have activity in modulating or inhibiting p38 MAP kinase activity. As such, it is anticipated that the compounds possessing such activity will be useful therapeutic agents in the prophylaxis or treatment of diseases where the disease or condition is one in which the activity of p38 MAP kinase initiates or facilitates development of the disease. Examples of conditions ameliorated by the inhibition of p38 MAP kinase are discussed above, and include, but are not limited to, rheumatoid arthritis, osteoarthritis, rheumatoid spondylitis, gouty arthritis, traumatic arthritis, rubella arthritis, psoriatic arthritis, and other arthritic conditions; Alzheimer's disease; toxic shock syndrome, the inflammatory reaction induced by endotoxin or inflammatory bowel disease; tuberculosis, atherosclerosis, muscle degeneration, Reiter's syndrome, gout, acute synovitis, sepsis, septic shock, endotoxic shock, gram negative sepsis, adult respiratory distress syndrome, cerebral malaria, chronic pulmonary inflammatory disease, silicosis, pulmonary sarcoisosis, bone resorption diseases, reperfusion injury, graft vs. host reaction, allograft rejections, fever and myalgias due to infection, such as influenza, cachexia, in particular cachexia secondary to infection or malignancy, cachexia secondary to acquired immune deficiency syndrome (AIDS), AIDS, ARC (AIDS related complex), keloid formation, scar tissue formation, Crohn's disease, ulcerative colitis, pyresis, chronic obstructive pulmonary disease (COPD), acute respiratory distress syndrome (ARDS), asthma, pulmonary fibrosis and bacterial pneumonia.

Of particular interest are compounds for use in the treatment or prophylaxis of inflammatory diseases and conditions, rheumatoid arthritis and osteoarthritis.

In another aspect, the invention provides the use of a compound of the formula (I) as defined herein for the manufacture of a medicament for the prophylaxis or treatment of a disease state or condition mediated by a p38 MAP kinase.

In a further aspect, the invention provides a method for the prophylaxis or treatment of a disease state or condition mediated by a p38 MAP kinase, which method comprises administering to a subject in need thereof a compound of the formula (I) as defined herein.

The invention also provides a method of inhibiting a p38 MAP kinase, which method comprises contacting the p38 MAP kinase with a kdnase-inhibiting compound of the formula (I) as defined herein.

The invention further provides a method of modulating a cellular process by inhibiting the activity of a p38 MAP kinase using a compound of the formula (I) as defined herein.

In the definition of the compounds of the formula (I) above and as used hereinafter, the term “hydrocarbyl” is a generic term encompassing aliphatic, alicyclic and aromatic groups having an all-carbon backbone. Examples of such groups include alkyl, cycloalkyl, cycloalkenyl, carbocyclic aryl, alkenyl, alkynyl, cycloalkylalkyl, cycloalkenylalkyl, and carbocyclic aralkyl, aralkenyl and aralkynyl groups. Such groups can be unsubstituted or substituted by one or more substituents as defined herein. The examples and preferences expressed below apply to each of the hydrocarbyl substituent groups or hydrocarbyl-containing substituent groups referred to in the various definitions of substituents for compounds of the formula (I) unless the context indicates otherwise.

Where reference is made to a hydrocarbyl group being “optionally interrupted” by one or more atoms or groups (e.g. by O, S, SO, SO₂, NH or N—C₁₋₄ hydrocarbyl in the case of the group R^{b}), this is intended to refer to the case in which one or more of the said atoms or groups is interposed between adjacent carbon atoms in the carbon backbone of the hydrocarbyl group. For example, according to this definition, a —CH₂—CH₂—O—CH₂—CH₂— group can be viewed as a butylene group interrupted by an oxygen atom.

Except where the context indicates otherwise, preferred aliphatic hydrocarbyl groups are those having from 1 to 8 carbon atoms, more typically from 1 to 6 carbon atoms, more preferably from 1 to 4 carbon atoms. Preferred alicyclic hydrocarbyl groups are those including up to 10 ring members, and more usually up to six ring members. Preferred aromatic carbocyclic groups are those having up to 10 ring members, more preferably up to 6 ring members.

The term “alkyl” covers both straight chain and branched chain alkyl groups. Unless the context indicates otherwise, the term “alkyl” refers to groups having 1 to 8 carbon atoms, and typically from 1 to 6 carbon atoms, for example from 1 to 4 carbon atoms. Examples of alkyl groups include methyl, ethyl, propyl, isopropyl, n-butyl, isobutyl, tert-butyl, n-pentyl, 2-pentyl, 3-pentyl, 2-methyl butyl, 3-methyl butyl, and n-hexyl and its isomers.

Examples of cycloalkyl groups are those having from 3 to 10 ring atoms, particular examples including those derived from cyclopropane, cyclobutane, cyclopentane, cyclohexane and cycloheptane, bicycloheptane and decalin.

Examples of alkenyl groups include, but are not limited to, ethenyl (vinyl), 1-propenyl, 2-propenyl (allyl), isopropenyl, butenyl, buta-1,4-dienyl, pentenyl, and hexenyl.

Examples of cycloalkenyl groups include, but are not limited to, cyclopropenyl, cyclobutenyl, cyclopentenyl, cyclopentadienyl and cyclohexenyl. Examples of alkynyl groups are those having from 2 to 8 carbon atoms, more typically from 2 to 6 carbon atoms, for example from 2 to 4 carbon atoms. Examples of alkynyl groups include, but are not limited to, ethynyl and 2-propynyl (propargyl) groups.

Examples of carbocyclic aryl groups include phenyl, naphthyl, indenyl, and tetrahydronaphthyl.

Examples of cycloalkylalkyl, cycloalkenylalkyl, carbocyclic aralkyl, aralkenyl and aralkynyl groups include phenethyl, benzyl, naphthylmethyl, styryl, phenylethynyl, cyclohexylmethyl, cyclopentylmethyl, cyclobutlymethyl, cyclopropylmethyl and cyclopentenylnethyl groups.

For substituents attached directly to the fused five membered and six membered rings, small alkyl groups are generally preferred, presently preferred groups including methyl and ethyl, with methyl being particularly preferred.

The term “aryl” as used herein (for example in the terms “arylamino” and “arylthio”), except where the context indicates otherwise, refers to a carbocyclic or heterocyclic group having aromatic character. The aryl group can be a monocyclic or bicyclic group and can be unsubstituted or substituted with one or more substituents. The term “aryl” embraces polycyclic (e.g. bicyclic) ring systems wherein one or more rings are non-aromatic, provided that at least one ring is aromatic. Examples of non-heterocyclic aryl groups include phenyl, indenyl, tetrahydronaphthyl and naphthyl, and such groups may be unsubstituted or substituted with one or more substituents. Examples of heterocyclic groups are those set out herein in relation to the group R³.

The term “monocyclic heterocyclic group” as used herein, except where the context dictates otherwise, refers to both aromatic and non-aromatic heterocyclic groups. Examples of aromatic heterocyclic groups are the monocyclic groups listed in respect of substituent group R³. Examples of non-aromatic heterocyclic groups include, but are not limited to, rings containing up to three heteroatoms selected from nitrogen, sulphur and oxygen. Typically at least one nitrogen atom will be present. Particular examples of such groups include piperidine, piperazine, N-methylpiperazine, morpholine, pyrrolidine, imidazoline, imidazolidine, thiazoline, thiazolidine, oxazoline, oxazolidine and tetrahydrofiran. Preferred non-aromatic heterocyclic groups include morpholine and piperidine, particularly morpholine.

The term “halogen” as used herein includes fluorine, chlorine, bromine and iodine, but fluorine and chlorine are generally preferred as substituents.

The compounds of the formula (I) are indoles or aza-indoles containing one, two or three nitrogen atoms in the six membered ring. Typically the six membered ring contains no more than two nitrogen atoms, and preferably no more than one. Indoles are particularly preferred.

In one embodiment, T and V are each a group CR⁴, and preferably at least one (e.g. U) and more preferably both of U and W are each a group CR⁴.

In another embodiment, one of U and W is a group CR⁴, and preferably T and V are also both CR⁴. For example U can be a group CR⁴ whilst W is a nitrogen atom, or both U and W can be CR⁴.

The group R⁴ can be hydrogen or a group Y or a relatively small substituent such as hydroxy, halogen, nitro, cyano, a monocyclic heterocyclic group having up to seven ring members, a group N(R⁵)₂, a group C(O)N(R⁶)₂, a group SO₂N(R⁶)₂ or a group R^{a}—R^{b} as hereinbefore defined. Only one group Y is typically present.

Thus, the six membered ring of the indole/azaindole nucleus can be unsubstituted or substituted. In one embodiment (for example when the compound is an indole), the six membered ring is unsubstituted or is substituted with up to two (for example one) small substituents selected from methyl, chlorine, amino, fluorine, nitro and acetamido.

For example, in one sub-group of compounds, V is CH and/or W is CH or C—CH₃ and/or U is selected from CH, C—CH₃, and fluorine and/or T is a carbon atom substituted by methyl, chloro, nitro or a group (R⁵)₂N as hereinbefore defined

In a further embodiment both of U and W can be a group CR⁴. Typically, V is CH.

Where R⁴ is a group R^{a}—R^{b}, the moiety R^{a} can be a chemical bond, or it can be O, S, SO, SO₂, NH or N—C₁₋₄ hydrocarbyl, and the group R^{b} can be C₁₋₈ hydrocarbyl optionally interrupted by O, S, SO, SO₂, NH or N—C₁₋₄ hydrocarbyl and optionally substituted by one or more substituents. Examples of hydrocarbyl groups and preferred hydrocarbyl groups are as set out above. In the context of the groups R^{a} and R^{b}, small allyl groups are particularly preferred, for example methyl groups. Optional substituent groups for R^{b} are selected from hydroxy, amino, mono- or di-C₁₋₄ hydrocarbylamino, C₁₋₄ hydrocarbyloxy, oxo, C₁₋₄ hydrocarbylthio and halogen. Small substituent groups such as C₁ groups and smaller halogens such as chlorine and fluorine are preferred.

Each group R⁵ in the optional group N(R⁵)₂ is independently selected from hydrogen, C₁₋₄ alkyl, C₁₋₄ acyl and C₁₋₄ alkylsulphonyl groups. Hydrogen and C₁ groups are preferred.

Each group R⁶, when present, is independently selected from hydrogen and C₁₋₄ hydrocarbyl, hydrogen and methyl being preferred.

One particular subset of compounds of the formula (I) is the set of compounds having a substituent group Y which is a group —N(R⁷)—C(O)—R⁸ or —N(R⁷)—SO₂—R⁸.

The group R⁷ can be hydrogen, C₁₋₄ hydrocarbyl or a group C(O)—R⁸ or SO₂—R⁸. Where it is C₁₋₄ hydrocarbyl, it is typically methyl.

R⁸ is selected from C₁₋₁₀ hydrocarbyl, C₁₋₁₀ hydrocarbylamino, C₁₋₁₀ hydrocarbylthio, C₁₋₁₀ hydrocarbyloxy, and aryl, arylamino, arylthio and aryloxy groups, the terms hydrocarbyl and aryl being as generally defined above.

In the context of the group Y, the aryl moieties can be carbocyclic or heterocyclic and have from five to twelve ring members. Carbocyclic aryl groups such as phenyl, or monocyclic heterocyclic groups containing one or two nitrogen atoms, are presently preferred. Each substituent group R⁸ can be unsubstituted or substituted by one or more groups R⁴ as hereinbefore defined (other than Y).

Thus, Y can take the form of an amide, carbamate, urea or thiourea compound.

Alternatively, R⁷ and R⁸ together with the nitrogen and carbon or sulphur atoms to which they are attached can be linked to form a ring structure of 4 to 7 ring members. Where R⁸ is an aryl, arylamino, arylthio or aryloxy group, it may be linked to R⁷ to form a fused bicyclic heterocyclic structure.

In one preferred sub group of compounds, R⁸ is selected from optionally substituted aryl, arylamino, arylthio and aryloxy, R⁸ typically being a carbocyclic or heterocyclic aryl, arylamino, arylthio or aryloxy group wherein the aryl moiety has five or six ring members. It is presently preferred that R⁸ is selected from unsubstituted aryl and arylamino groups, and substituted aryl and arylamino groups wherein the aryl group is phenyl or a five or six-membered heterocyclic group having one or two nitrogen ring members, for example a group selected from pyridyl, pyrazolyl and isoxazolyl groups. Particularly preferred aryl groups are phenyl, pyridyl (e.g. 4-pyridyl) and pyrazolyl (e.g. 2-pyrazolyl).

For example, the aryl (e.g. phenyl, pyridyl or pyrazolyl) ring can be substituted by one or more substituents selected from halogen, a monocyclic heterocyclic group having up to seven ring members and a group R^{a}—R^{b}. Preferred substituents are fluorine, chorine, methoxy, trifluoromethoxy, trifluoromethyl, methyl, ethyl, isopropyl, isobutyl, t-butyl, phenyl, and five and six membered monocyclic heterocyclic groups. When the aryl group is a pyrimidinyl group, particularly a 2-pyrimidinyl group, it is preferred that the aryl group is not substituted by phenyl. Most preferably the aryl group is other than 5-phenylpyrimidin-2-yl.

In one preferred form, the aryl group is a phenyl ring containing one or two meta substituents, for example wherein one meta position on the phenyl ring is unsubstituted or is substituted by a group selected from fluorine, chorine, methoxy, trifluoromethoxy, trifluoromethyl, ethyl, methyl and isopropyl; and the other meta position is substituted by a group selected from fluorine, chorine, methoxy, trifluoromethoxy, trifluoromethyl, ethyl, methyl, isopropyl, isobutyl, t-butyl, phenyl, substituted phenyl, and five and six membered monocyclic heterocyclic groups.

In a particular sub group of compounds, the phenyl ring contains a single substituent which is selected from m-trifluoromethyl and m-trifluoromethoxy. Alternatively, the phenyl ring can bear a fluoro substituent at one meta-position and a morpholino group at the other meta-position.

In another preferred sub-group of compounds, the aryl ring is a pyridyl ring, such as a 4-pyridyl ring, substituted by a five or six membered monocyclic heterocyclic group such as morpholino.

In a further preferred sub-group of compounds, the aryl ring is a pyrazolyl or isoxazolyl (preferably pyrazolyl) group substituted by a phenyl group and/or a C₁₋₄ hydrocarbyl group, particularly a C₁₋₄ alkyl group, and most preferably a tertiary butyl group. A 2-phenyl-5-t-butylpyrazol-3-yl group has been found to be particularly advantageous.

The five membered ring of the compounds of the formula (I) is linked via a linker group A to a heteroaryl group R³. The linker group has a linking chain length of one or two atoms: in other words the number of atoms in the backbone of the linker group is one or two. Thus, for example, a group —CH₂— has a liking chain length of one, whilst a group —CH₂—CH₂— has a linking chain length of two.

Examples of linker groups A include CH₂, C═O, O, S, SO, SO₂, NR′, CHR, CR₂, CR₂CR₂, CR═CR, OCH₂, CH₂O, CH₂S, SCH₂, SOCH₂, CH₂SO, SO₂CH₂, CH₂SO₂, NR′CH₂, CH₂NR′, CONR′, R′NCO, SO₂NR′, NR′SO₂, COCH₂ and CH₂CO, wherein R, where present, is independently selected from hydrogen, methyl and fluoro, and R′ where present is independently selected from hydrogen and methyl. Presently preferred linker groups A include CH₂ or CH₂CH₂, the ethylene group being particularly preferred.

The heteroaryl group R³ is a monocyclic or bicyclic group containing from five to twelve ring members, and more usually from five to ten ring members. The hereoaryl group can be, for example, a five membered or six membered monocyclic ring or a bicyclic structure formed from fused five and six membered rings or two fused six membered rings. Each ring may contain up to about four heteroatoms, more usually three or fewer, and typically one, two or three. The heteroatoms are typically selected from nitrogen, sulphur and oxygen. In one embodiment, the heteroaryl ring contains at least one ring nitrogen atom. The nitrogen atoms in the heteroaryl rings can be basic, as in the case of a pyridine or pyrimnidine, or essentially non-basic as in the case of an indole or pyrrole nitrogen. In general the number of basic nitrogen atoms present in the heteroaryl group, including any amino group substituents of the ring, will be less than five.

Examples of heteroaryl groups R³ include but are not limited to pyridyl, pyrrolyl, filranyl, thienyl, imidazolyl, oxazolyl, oxadiazolyl, oxatriazolyl, isoxazolyl, thiazolyl, isothiazolyl, pyrazolyl, pyrazinyl, pyrimidinyl, triazinyl, quinolinyl, isoquinolinyl, tetrazolyl, benzfuranyl, chromanyl, thiochromanyl, benzimidazolyl, benzoxazolyl, benzisoxazole, benzthiazolyl and benzisothiazole, isobenzofuranyl, isoindolyl, indolizinyl, indolinyl, isoindolinyl, purinyl (e.g., adenine, guanine), indazolyl, benzodioxolyl, chromenyl, isochromenyl, chroman, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl.

It is presently preferred that the group R³ is a monocyclic heteroaryl group containing at least one nitrogen atom, and one particular example of such a group is pyridyl, for example a 4-pyridyl group.

The group R³ can be unsubstituted or substituted by one or more groups selected from halogen, nitro, cyano, a monocyclic heterocyclic group having up to seven ring members, a group N(R⁵)₂, a group C(O)N(R⁶)₂, a group SO₂N(R⁶)₂, and a group R^{a}—R^{b}; wherein R⁵, R⁶, R^{a} and R^{b} are as hereinbefore defined.

In one sub-group of compounds, R³ is unsubstituted.

In another sub-group of compounds, R³ is substituted.

Where substituents are present on the heteroaryl ring, examples of substituents include but are not limited to C₁₋₆ alkyl, C₁₋₆ alkoxy, amino, C₁₋₆ alkylamino, di-C₁₋₆ alkylamino, halogen, hydroxy, trifluoromethyl, cyano, nitro, C₂₋₆ alkenyl, C₂₋₆ alkynyl, C₁₋₆ alkylthio, amino C₁₋₆ alkyl, hydroxy C₁₋₆ alkyl, C₁₋₄ alkoxyalkyl, phenyl-C₁₋₆ alkyl, hydroxyalkylamino, aminoalkylamino and aminoalkoxy, C₃₋₇ cycloalkyl and monocyclic C₅₋₆ carbocyclic or heterocyclic groups containing up to three heteroatoms. Particular examples of substituents include chlorine, fluorine, methyl, unsubstituted amino, 2-hydroxyethylamino, 2-hydroxyprop-2-ylamino, 2-hydroxy-2-methylprop-2-ylamino, 1-phenylethyl, morpholino and piperazino groups.

Where two or more substituents (“larger substituents”) each having a chain length of greater than three atoms are present on the heteroaryl group R³, it is preferred that they are located on the “same side” of the ring. In other words, where for example three such larger substituents are present on a six membered ring, it is preferred that they are located at adjacent ortho, meta and para positions, relative to the point of attachment to the group A. Where two such larger substituents are present, it is preferred that they are located on adjacent ortho and meta positions, or adjacent meta and para positions, or adjacent (spaced apart by one ring position) ortho and para positions. The term “chain length” in the present context refers to the number of atoms extending in a continuous chain outwardly from the heteroaryl ring. Thus, for example, a chlorine substituent has a chain length of one, a methyl group has a chain length of two, and an ethyl group has a chain length of three. “Smaller substituents”, i.e. substituents having a chain length of three or less, may be present on one or both “sides” of the ring, whether or not “larger” substituents are also present.

It is preferred that the group -A-R³ contains no carboxylic, phosphonic and sulphonic acid groups, nor any tetrazole or —C(O)NSO₂— groups.

It is also preferred that when -A- is —C(O)N—, the nitrogen atom of the group A is not linked directly to a furan or thiophene ring.

It is preferred that when in combination, U, T, V and W are all CH, R¹ and R² are both hydrogen, and R⁰ is a group —CH₂—CH₂—R3, R³ is other than a pyrazin-3-yl or pyrid-3-yl group.

The group -A-R³ can be attached to either the 1-position or the 3-position of the five membered ring, preferably the 3-position.

When -A-R³ is attached to the 3-position (i.e. R⁰ is -A-R³), it is preferred that R¹ is hydrogen or methyl, particularly hydrogen.

When -A-R³ is attached to the 1-position (i.e. R¹ is -A-R³), it is preferred that R⁰ is hydrogen or methyl, particularly hydrogen.

When -A-R³ is attached to the 3-position (i.e. R¹ is -A-R³), and a group Y is present, the group Y is advantageously located at the 5-position of the bicyclic (e.g. indole) group.

When -A-R³ is attached to the 1-position (i.e. R¹ is -A-R³), and a group Y is present, the group Y is advantageously located at the 6-position of the bicyclic (e.g. indole) group.

The group R² is typically a small substituent and preferably is selected from hydrogen and methyl. Most typically, R² is hydrogen.

Novel Compounds

Many of the compounds of the formula (I) are novel. In a further aspect, therefore, the invention provides novel compounds per se of the formula (I). One group of novel compounds within the scope of the present invention is the group of compounds of the formula (I) as hereinbefore defined but provided that one group R⁴ is a group Y, and excluding the known compound wherein in combination R¹ and R² are hydrogen, U, V and W are all CH and T is a carbon atom bearing an unsubstituted benzamido group.

In the novel compounds of the invention, it is most preferred that the group -A-R³ contains no carboxylic, phosphonic and sulphonic acid groups, nor any tetrazole or —C(O)NSO₂— groups. It is also preferred that when -A- is —C(O)N—, the nitrogen atom of the group A is not linked directly to a furan or thiophene ring.

It is fiurther preferred, in respect of the novel compounds of the formula (I), that R¹ is H or methyl.

One sub-group of novel compounds is the group of compounds of the formula (I) wherein either T or V (preferably T) is a group C—Y, wherein Y is a group —N(R⁷)—C(O)—R⁸ or —N(R⁷)—SO₂—R⁸ as hereinbefore defined.

Within this sub-group of compounds is the group of compounds per se wherein R⁸ is selected from carbocyclic or heterocyclic aryl, arylamino, arylthio and aryloxy groups wherein the aryl moiety has five or six ring members (but excluding the known unsubstituted benzamido compound referred to above) and R⁷ is hydrogen or C₁₋₄ hydrocarbyl (preferably hydrogen or methyl).

One group of preferred novel compounds per se is the group in which the aryl moiety is carbocyclic, for example wherein R⁸ is selected from unsubstituted phenyl and phenylamino groups, and substituted phenyl and phenylamino groups.

In another preferred group of novel compounds, the aryl moiety is a five or six membered heterocylic group having one or two nitrogen ring members, for example a pyridyl or pyrazolyl group.

Particular novel compounds of the invention are compounds wherein the phenyl, pyridyl or pyrazolyl ring is substituted by one or more substituents selected from halogen, a monocyclic heterocyclic group having up to seven ring members and a group R^{a}—R^{b} as hereinbefore defined. Particular examples of substituents are selected from fluorine, chorine, methoxy, trifluoromethoxy, trifluoromethyl, methyl, ethyl, isopropyl, isobutyl, t-butyl; phenyl, and five and six membered monocyclic heterocyclic groups.

One sub-group of compounds per se is the group of compounds wherein the phenyl ring contains one or two meta substituents, for example wherein one meta position on the phenyl ring is unsubstituted or is substituted by a group selected from fluorine, chorine, methoxy, trifluoromethoxy, trifluoromethyl, ethyl, methyl and isopropyl; and the other meta position is substituted by a group selected from fluorine, chorine, methoxy, trifluoromethoxy, trifluoromethyl, ethyl, methyl, isopropyl, isobutyl, t-butyl, phenyl, substituted phenyl, and five and six membered monocyclic heterocyclic groups.

Examples of particularly preferred novel compounds of the invention are those wherein the phenyl ring contains a single substituent which is selected from m-trifluoromethyl and m-trifluoromethoxy groups.

Further examples of particularly preferred compounds are those wherein the aryl ring is a pyrazolyl ring substituted by a phenyl group and a tert-butyl group. In another group of novel compounds, the compound is an indole in which R⁰ is -A-R³, wherein A is ethylene and R³ is a pyrimidinyl group substituted at the 2-position by a hydroxyalkylanino group or a phenylethyl group.

Specific examples of novel compounds within the scope of the present invention include:
3-(2-(4-pyridyl)ethyl)-5-(3-trifluoromethoxybenzamido)indole;
3-(2-(4-pyridyl)ethyl)-5-(3-trifluoromethylbenzamido)indole;
3-(2-(4-pyridyl)ethyl)-5-(3-fluoro-5-(1-N-morpholino)benzamido)indole;
1-(2-(4-pyridyl)ethyl)-5-(3-fluoro-5-(1-N-morpholino)benzamido)indole;
5-(phenylcarbamoylamino)-3-(2-(4-pyridyl)ethyl)indole;
5-(3-tert-butyl-1-phenylpyrazol-5-ylcarbamoylamino)-3-(2-(4-pyridyl)ethyl)indole;
3-2-(2-(2-hydroxyethylamino)-4-pyrimidinyl)ethyl)indole;
3-(2-(2-(3-hydroxy-2-methyl-prop-2-ylamino)-4-pyrimidinyl)ethyl)indole;
3-(2-(2-((S)-(−)-α-methylbenzylamino)-4-pyrimidinyl)ethyl)indole;
3-(2-(2-((S)-(+)-α-methylbenzylamino)-4pyrimidinyl)ethyl)indole;
6-(3-fluoro-5-(4-morpholino)benzamido)-3-(2-(4-pyridyl)ethyl)indole; and
6-(3-fluoro-5-(4-morpholino)benzamido)-1-(2-(4-pyridyl)ethyl)indole.

In a further aspect, the invention provides novel compounds of the formula (I) as hereinbefore defined for use in medicine and pharmaceutical compositions comprising a novel compound of the formula (I) in association with a pharmaceutically acceptable carrier.

Many compounds of the formula (I) can exist in the form of salts, for example acid addition salts or, in certain cases salts of organic and inorganic bases such as carboxylate, sulphonate and phosphate salts. All such salts are within the scope of this invention, and references to compounds of the formula (I) include the salt forms of the compounds.

Acid addition salts may be formed with a wide variety of acids, both inorganic and organic. Examples of acid addition salts include salts formed with hydrochloric, hydriodic, phosphoric, nitric, sulphuric, citric, lactic, succinic, maleic, malic, isethionic, fumaric, benzenesulphonic, toluenesulphonic, methanesulphonic, ethanesulphonic, naphthalenesulphonic, valeric, acetic, propanoic, butanoic, malonic, glucuronic and lactobionic acids.

Compounds of the formula may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I).

Also encompassed by formula (I) are any polymorphic forms of the compounds, solvates (e.g. hydrates), complexes (e.g. inclusion complexes or clathrates with compounds such as cyclodextrins, or complexes with metals) of the compounds, and pro-drugs of the compounds. By “prodrugs” is meant for example any compound that is converted in vivo into a biologically active compound of the formula (I).

Where the compounds of the formula (I) contain chiral centres, all individual optical forms such as enantiomers, epimers and diastereoeisomers, as well as racemic mixtures of the compounds are within the scope of formula (I).

Methods for the Preparation of Compounds of the Formula (I)

Compounds of the formula (I) can be prepared in accordance with methods known per se or as described herein. For example, compounds of the formula (I) wherein the group A-R³ is attached to the 3-position of the five membered ring can be prepared in accordance with methods similar or analogous to those described in US patent numbers U.S. Pat. No. 3,300,506 and U.S. Pat. No. 3,409,626, the disclosures of each of which are incorporated herein by reference. Thus, compounds of the formula (I) bearing a group A-R³ at the 3-position of the five membered ring can be prepared by reacting a compound of the formula (II): 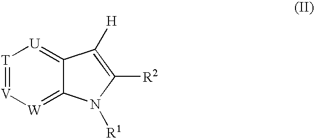
wherein U, T, V, W and R² are as defined in respect of formula (I) and R¹ is hydrogen or a C₁₋₄ alkyl group with an electrophilic group capable of introducing the intact group A-R³ or a precursor thereof (e.g. a protected form). When A is an ethylene group, the compound of the formula (II) can be reacted with a vinyl compound H₂C═CH—R³, for example in the presence of an acid such as acetic acid. The reaction is typically conducted at an elevated temperature, for example at a temperature of above 75° C., more usually above 100° C., for example at approximately 130° C. The resulting product can be purified in the usual manner by means of chromatography.

Compounds of the formula (I) in which the linker group is CH, CR, C(O), S(O), S(O₂) or C(O)CH₂ and is attached to the 3-position of the five membered ring can be prepared by electrophilic substitution, for example by means of a Friedel Crafts-type reaction, of a compound of the formula (II) as defined above. Reagents for effecting electrophilic substitution can talce the form R³-A′-X wherein X is a suitable leaving group such as a halogen (e.g. chlorine) and A′ is selected from CH, CR, C(O), S(O), S(O₂) and C(O)CH₂.

Alkylation at the 3-position can also be carried out by reacting an appropriately substituted 3-unsubstituted indole compound of the formula R³-A′-X wherein A′ is methylene or ethylene and X is a leaving group such as bromine in the presence of silver (I) oxide in a polar solvent such as dioxan, under conditions similar or analogous to those described in WO99/43654.

Compounds of the formula (I) in which the linker group is —CO— can be prepared by formation of 3-indole organometallic reagents (e.g. Grignard) from the corresponding 3-halogen substituted indole (e.g. iodine) and then treatment with the appropriate R³ heterocycle acid chloride under conditions similar or analogous to those described in Indian J. Chem., 24 B(10), 1012-14, 1985. Indole-3-halogens can be obtained from commercial sources or can be prepared by known methods.

Compounds of the formula (I) in which the linker group is —CH₂CO— can be prepared by reacting a suitably N−1 protected derivative of an indole or azaindole acetic acid ester with a strong base in the presence of the appropriately substituted heterocyclic ester (e.g. methyl 4-pyridyl carboxylate), followed by hydrolysis and decarboxylation under conditions similar or analogous to those described in Khim. Geterotsikl. Soedin., (1), 55-58, 1980. Indole carboxylic acids can be obtained from commercial sources (for example indole-3-acetic acid) or can be prepared by known methods. Indole or aaaindole carboxylic acids or their reactive derivatives can also be used to prepare compounds in which the linker group is CONH by reaction with an appropriate amino substituted heterocyclic group R³.

Compounds wherein the linker is a group OCH₂ or SCH₂ can be prepared from appropriately substituted N-protected indoles bearing a hydroxyl group or SH group at the 3-position by reaction with a compound R³—CH₂—Br under conditions similar or analogous to those described in J. Med. Chem., 32(6),1360-6; 1989. Such reactions can be carried out in a polar solvent such as dimethylformamide (DMF) in the presence of a base such as sodium hydride. In certain cases, it may be desirable for the 3-hydroxy indole to be substituted at the 2-position by an ester group (for example methoxycarbonyl) so as to assist O-or S-alkylation. The ester group can thereafter be reduced to a methyl group to give a compound of the formula (I) wherein R² is methyl or hydrolysed to the carboxylic acid and removed by decarboxylation to give a compound of the formula (I) wherein R² is hydrogen.

Compounds wherein the linker group is a group CH₂O, CH₂S, CH₂NH or CH₂NMe can be prepared from an appropriately N-protected indole bearing a group CH₂NMe₂ at the 3-position. Methylation of the dimethylamrino group to form a quaternary ammonium compound and displacement of trimethylamine from the quaternary ammonium compound by reaction with an oxygen, sulphur or amino nucleophile suitable for introducing the group OR³, SR³, NHR₃ or NMeR³ gives the desired product. The methylation reaction can be effected in standard fashion by reaction with methyl iodide in a solvent such as benzene, for example under the conditions described in Tetrahedron Letters, 36(33), 5929-32; 1995. Indoles bearing a CH₂NMe₂ group at the 3-position can be prepared from the corresponding 3-formyl compound by a standard reductive alkylation using, for example, dimethylanmine and sodium cyanoborohydride. Alternatively, an appropriately substituted indole 3-carboxylic acid methyl or ethyl ester can be subjected to a hydride reduction to give the 3-hydroxymethyl derivative and then converted to the dimethylamino group in known fashion.

Compounds wherein the linker group A is CH═CH can be prepared by means of a Heck-type reaction between a compound of the formula R³—CH═CH₂ or by means of a Stille-type reaction with the tributyltin analogue R³—CH═CH—SnBu₃ and an appropriately substituted 3-haloindole (e.g. a 3-bromoindole) in the presence of palladium(0) under standard conditions or conditions analogous thereto.

Alternatively, compounds wherein the linker group is CH═CH can be prepared by reduction of an indole bearing a substituent group R³—CH(Cl)—C(O)— at its 3-position using a metal hydride reducing agent such as lithium aluminium hydride according to conditions similar or analogous to those described in Tetrahedron, 31(17), 2063-73; 1975.

In a further method of preparing compounds wherein the linker group is CH═CH, an N-protected indole or aza-indole bearing a 3-CHO group can be reacted under Wittig-type conditions with a triphenyl(arylmethyl)phosphonium compound suitable for introducing the group R³. The N-protecting group can be, for example, a phenylsulphonyl group. Such reactions are typically carried out under anhydrous conditions at low temperature in a polar non-protic solvent such as tetrahydrofuran.

Compounds wherein the linker group A is SO₂CH₂ or SOCH₂ can be prepared by reaction of an appropriately substituted indole with a sulphonylating agent such as R³CH₂SO₂Cl.

Compounds wherein the linker group A is SO₂NR can be prepared by reacting 3-indolylsulphonyl chlorides with an amine of the formula R³NH₂ or R³NH₂Me, optionally in the presence of another base, for example under conditions similar or analogous to those described in Buyanov et al, Khim. Geterotsikl. Soedin (1996), (1), 40-42 or as described in WO00/73264.

Compounds wherein the linker group A is NHSO₂ can be prepared by reacting an azide compound of the formula R³SO₂N₃ with a suitably 1-protected 3-unsubstituted indole, for example in a polar solvent such as dimethylsulphoxide (DMSO), under conditions similar or analogous to those described in J. Chem. Soc., Perkin Transactions 1, (8), 1688-92; 1980. Alternatively, compounds wherein the linker group A is NHSO₂ can be prepared by reacting a 3-amino indole with R³SO₂Cl, for example under conditions similar or analogous to those described in Khim. Geterotsikl. Soedin., (4), 481-5; 1977.

Compounds wherein the linker group A is NRCH₂ can be prepared reacting an appropriately substituted 1-acyl-3-oxindole with a compound R³CH₂NH₂ or R³CH₂NHMe, for example under conditions similar or analogous to those described in Khim Geterotsikl. Soedin., (7), 939-43; 1978. Alternatively, compounds wherein the linker A is a group is NRCH₂ can be prepared by reduction of a compound wherein A is NHCO with a metal hydride reducing agent such as LiAlH₄.

Compounds wherein the linker group A is NHCO can be prepared from an appropriately substituted 3-acylindole having a substituent R³C(O) at the 3-position by reaction with hydroxylamine to form the corresponding oxime followed by a Beckianan rearrangement, for example under conditions similar or analogous to those described in J. Chem., Res. Synop., (1), 4-5; 1983, to give the amide. The 3-acyl indoles can be prepared by Friedel Crafts acylation of the 3-unsusbtituted indole by reaction with the appropriate acid halide. Alternatively, compounds wherein the linker group A is NHCO can be prepared from a 3-amino 2-ethoxycarbonyl indole, for example under conditions similar or analogous to those described in J. Heterocycl. Chem., 24(2), 437-9; 1987, followed by reduction of the ester group to give the 2-methyl compound or hydrolysis and decarboxylation to give the 2-unsubstituted compound.

Compounds wherein the linker group A is S can be prepared by reaction of a aryl substituted hydrazine of the formula ArNHNH₂, wherein Ar is a substituted or unsubstituted phenyl group, with a compound of the formula R²—C(O)—CH₂—S—R³ to form a hydrazone and then cyclising the hydrazone in the presence of an acid such as acetic acid to give the desired compound. The reaction can be carried out under conditions similar or analogous to those described in Synthesis, (3), 270-2; 1994.

Compounds of the formula (I) containing a group CR⁴ wherein R⁴ is a substituted amino group can be prepared from the corresponding amino-indole or amino-aza-indole compounds. For example, when the amino group is substituted by an acyl group such as a benzoyl or substituted benzoyl group, the compound can be prepared by acylation of the corresponding amino compound. Such acylation reactions can be conducted in a polar solvent (such as dimethyl formamide or dimethylsulphoxide) in the presence of an acylation catalyst such as hydroxybenzotriazole, typically at a non-extreme temperature such as room temperature. The acylation of the amino group on the six membered ring of the fused ring system can be carried out before or after introducing the group R³.

When the amino group R⁴ group is substituted by a carbamoyl group, for example an optionally substituted phenylcarbamoyl group, the carbamoyl group can be introduced by reacting the corresponding amino analogue with an isocyanate such as an optionally substituted phenyl isocyanate. Reaction with an isocyanate can be carried out in a solvent, for example a chlorinated solvent such as chloroform or dichloromethane, at a moderately elevated temperature, for example between 60° C. and 100° C.

Compounds wherein the group R⁴ is an amino group can be prepared by reduction of the corresponding nitro-substituted compound. The reducing agent will generally be chosen so that it brings about reduction of the nitro group but not any heterocyclic group R³ that may be present. An example of a suitable reducing agent is an Fe/Fe(II) mixture which can be employed in a suitable polar solvent such as a dioxane, at a moderately elevated temperature between 60° C. and 100° C. (for example at around 90° C.).

Compounds of the formula (I) having a group A-R³ attached to the 1-position in the five membered ring, can be prepared from compounds of the formula (II) as hereinbefore defined wherein both of the 2- and 3-positions on the five membered ring are unsubstituted or substituted by a group R², and R¹ is hydrogen, by reaction with a suitable alkylating or acylating agent, optionally in the presence of a base. For example, compounds wherein the linker group A is an ethylene group can be prepared by reacting a 1-N-unsubstituted indole or azaindole with a vinyl heterocycle (R³CH═CH₂) in the presence of metal such as sodium and a copper reagent such as copper sulphate. Compounds having a substituent A-R³ at the 1-position can also be prepared by reacting a 1-N-unsubstituted compound with a compound L-A-R³, where L is a leaving group (such as a halide), in the presence of a strong base such as an alkali metal, an alkali metal hydride or hydroxide or an organometallic reagent such as an alkyl lithium.

Compounds of the formula (I) can also be prepared from other compounds of the formula (I) by functional group interconversions or by reaction with appropriate reagents in known manner.

In many of the synthetic schemes used to prepare compounds of the formula (I), the indole 1-position is protected in order to prevent it from taking part in the reaction. The protecting group used can be a simple alkyl group such as methyl, thereby leading directly to a compound of the formula (I) wherein R¹ is alkyl. Alternatively, however, the protecting group may be a removable protecting group such as an acyl group, a phenylsulphonyl group or a trialkylsilyl group such as tri-isopropylsilyl. Such protecting groups can be removed at an appropriate point in the reaction sequence by methods well known per se, for example using fluoride ion in the case of a silyl protecting group. Examples of protecting groups are described in the references set out above, and also in, for example, Protective Groups in Organic Synthesis (T. Green and P. Wuts; 3rd Edition; John Wiley and Sons, 1999).

Novel Chemical Intermediates

Certain of the key intermediates useful in preparing compounds of the formula (I) are novel compounds. Accordingly, in a further aspect, the invention provides novel compounds of the formula (III): 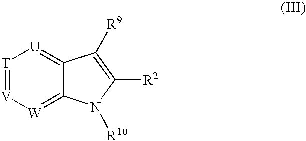
wherein U, T, V, W and R² are as hereinbefore defined in respect of the novel compounds of the formula (I); R⁹ is hydrogen, C₁₋₄ hydrocarbyl or halogen and R¹⁰ is hydrogen or C₁₋₄ hydrocarbyl, provided that at least one of R⁹ and R¹⁰ is hydrogen.

Particular groups of novel intermediates are the compounds corresponding to the preferred novel compounds per se as hereinbefore defined, but lacking the -A-R³ group.

Specific intermediate compounds believed to be novel include:
5-(3-trifluoromethoxybenzamido)indole;
5-(3-trifluoromethylbenzamido)indole;
5-(3-fluoro-5-(1-N-morpholino)benzamido)indole;
5-(3-fluoro-5-(1-N-morpholino)benzamido)indole;
5-(phenylcarbamoylamino)indole; and
5-(2-(4-morpholino)isonicotinamido))indole.
   Pharmaceutical Formulations

The invention also provides compounds of the formula (I) as hereinbefore defined in the form of pharmaceutical compositions.

The pharmaceutical compositions can be in any form suitable for oral, parenteral, topical, intranasal, intra-articular, ophthalmic, otic, rectal, intra-vaginal, or transdermal administration, or administration by inhalation. Where the compositions are intended for parenteral administration, they can be formulated for intravenous, intramuscular or subcutaneous administration.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Pharmaceutical compositions containing compounds of the formula (I) can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a celluloses or derivative thereof such as methyl cellulose, ethyl cellulose, hydroxypropyl methyl cellulose, and starches such as corn starch. Tablets may also contain such standard ingredients as binding and granulating agents agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (e.g. tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastrointestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract.

Compositions for topical use include ointments, creams, sprays, patches, gels, liquid drops and inserts (for example intraocular inserts). Such compositions can be formulated in accordance with known methods.

Compositions for parenteral and intra-articular administration are typically presented as sterile aqueous or oily solutions or fine suspensions, or may be provided in finely divided sterile powder form for maling up extemporaneously with sterile water for injection.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the inventions will generally be presented in unit dosage form and, as such, will typically contain sufficient compound to provide a desired level of biological activity. For example, a formulation intended for oral administration may contain from 0.1 milligrams to 2 grams of active ingredient, more usually from 10 milligrams to 1 gram, for example, 50 milligrams to 500 milligrams. The active compound will be administered to a patient in need thereof (for example a human or animal patient) in an amount sufficient to achieve the desired therapeutic effect.

Methods of Treatment

It is envisaged that the compounds of the formula (I) will useful in the prophylaxis or treatment of a range of disease states or conditions mediated by p38 MAP kinases. Examples of such disease states and conditions are set out above.

Compounds of the formula (I) are generally administered to a subject in need of such administration, for example a human or animal patient, preferably a human. The compounds will typically be administered in amounts that are therapeutically or prophylactically useful and which generally are non-toxic. However, in certain situations (for example in the case of life threatening diseases), the benefits of administering a compound of the formula (I) may outweigh the disadvantages of any toxic effects or side effects, in which case it may be considered desirable to administer compounds in amounts that are associated with a degree of toxicity.

A typical daily dose of the compound can be in the range from 100 picograms to 10 milligrams per kilogram of body weight, more typically 10 nanograms to 1 milligram per kilogram of bodyweight although higher or lower doses may be administered where required. Ultimately, the quantity of compound administered will be commensurate with the nature of the disease or physiological condition being treated and will be at the discretion of the physician.

The compounds of the formula (I) can be administered as the sole therapeutic agent or they can be administered in combination therapy with one of more other compounds for treatment of a particular disease state, for example rheumatoid arthritis and osteoarthritis. Examples of other therapeutic agents that may be administered together (whether concurrently or at different time intervals) with the compounds of the formula (I) include methotrexate, prednisilone, sulfasalazine, leflunomide and NSAIDs, for example COX-2 inhibitors such as celecoxib and rofecoxib.

### EXAMPLES

The invention will now be illustrated, but not limited, by reference to the specific embodiments described in the following examples.

### Example 1

### 3-(2-(4-Pyridyl)ethyl)indole

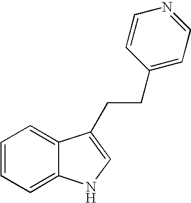

A mixture of 4-vinylpyridine (1.0 mmol) and indole (1.0 mmol) in acetic acid (1 ml) were stirred and at 130° C. for 16 hours. Upon cooling to room temperature the solvent was removed under reduced pressure and the residue subjected to purification by flash chromatography on silica gel. Elution with ethyl acetate or 10% methanol in ethyl acetate afforded the title compound.

By substituting the appropriate vinyl substituted monoheterocycle for vinyl pyridine and using the appropriate indole or azaindole, the method of Example 1 was used to prepare the following compounds.

### Example 2

### 1-Methyl-3-(2-(4-pyridyl)ethyl)indole

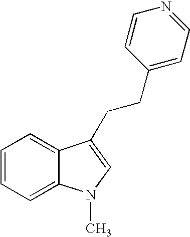

From 4-vinylpyridine and 1-methylindole; from SPECS (product code AE-473/30364014)

### Example 3

### 3-(2-(2-Pyridyl)ethyl)indole

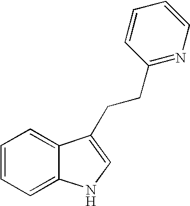

From 2-vinylpyridine and indole; from Salor (product code S64,176-6).

### Example 4

### 5-Methyl-3-(2-(4-pridyl)ethyl)indole

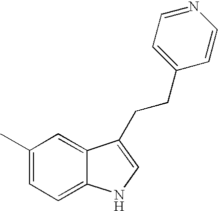

From 4-vinylpyridine and 5-methylindole; δ_{H} (400 MHZ, CDCl₃) 8.48 (2H, d, J 6), 7.97 (1H, br s), 7.37 (1H, s), 7.26 (1H, d, J 8), 7.17 (2H, d, J 6), 7.04 (1H, d, J 8), 6.82 (1H, d, J 2), 3.06 (4H, m), 2.47 (3H, s).

### Example 5

### 5-Chloro-3-(2-(4-pyridyl)ethyl)indole

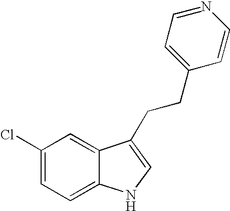

From 4-vinylpyridine and 5-chloroindole; δ_{H} (400 MHz, CDCl₃) 8.48 (2H, d, J 5.5), 8.10 (1H, br s), 7.56 (1H, d, J 2), 7.28 (1H, d, J 8.5), 7.15 (1H, dd, J 8.5, 2), 7.12 (2H, d, J 5.5), 6.88 (1H, d, J2), 3.03 (4H, m).

### Example 6

### 5-Nitro-3-(2-(4-pyridyl)ethyl)indole

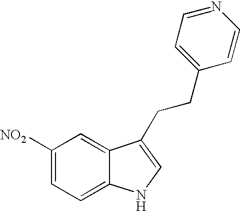

From 4-vinylpyridine and 5-nitroindole; δ_{H} (400 MHz, d₆-DMSO) 11.58 (1H, br s), 8.54 (1H, d, J 2), 8.44 (2H, d, J 6), 7.98 (1H, dd, J 9, 2), 7.50 (1H, d, J 9), 7.39 (1H, s), 7.30 (2H, d, J 6), 3.12 (2H, t, J 8), 3.00 (2H, t, J 8).

### Example 7

### 5-Acetamido-3-(2-(4-pyridyl)ethyl)indole

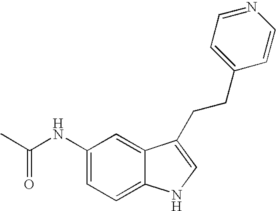

From 4-vinylpyridine and 5-aminoindole; δ_{H} (400 M, d₆-DMSO) 10.68 (1H, br s), 9.73 (1H, br s), 8.44 (2H, d, J 6), 7.85 (1H, s), 7.26 (2H, d, J 6), 7.21 (1H, d, J 8.5), 7.18 (1H, dd, J 8.5, 2), 7.05 (1H, d, J 2), 2.96 (4H, s), 2.02 (3H, s).

### Example 8

### 2-Methyl-3-(2-(4-pyridyl)ethyl)indole

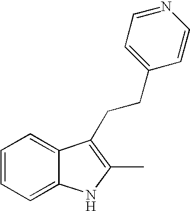

From 4-vinylpyridine and 2-methylindole; δ_{H} (400 MHz, CDCl₃) 8.48 (2H, d, J 6), 7.85 (1H, br s), 7.48 (1H, d, J 7.5), 7.29 (1H, d, J 7), 7.11 (4H, m), 3.00 (4H, m), 2.07 (3H, s).

### Example 9

### 4-Methyl-3-(2-(4-pyridyl)ethyl)indole

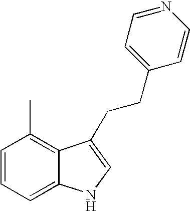

From 4-vinylpyridine and 4-methylindole; δ_{H} (400 Mz, CDCl₃) 8.50 (2H, d, J 6), 8.03 (1H, br s), 7.21 (1H, d, J 8),7.14 (2H, d, J 6),7.08 (1H, t, J 7.5),6.87 (1H, d, J 7.5), 6.84 (1H, d, J 2), 3.26 (2H, t, J 8.5), 3.01 (2H, t, J 8.5), 2.75 (3H, s).

### Example 10

### 7-Methyl-3-(2-(4-pyridyl)ethyl)indole

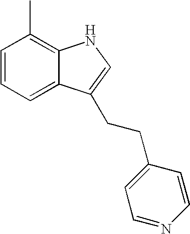

From 4-vinylpyridine and 7-methylindole; δ_{H} (400 MHz, CDCl₃) 8.52 (2H, d, J 6), 7.95 (1H, br s), 7.47 (1H, d, J 8), 7.12 (2H, d, J 6), 7.07 (1H, dd, J 8, 7), 7.02 (1H, d, J 7), 6.89 (1H, d, J2.5), 3.06 (4H, m), 2.49 (3H, s).

### Example 11

### 4-Fluoro-3-(2-(4-pyridyl)ethyl)indole

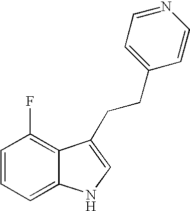

From 4-vinylpyridine and 4-fluoroindole; δ_{H} (400 MHz , d₆-DMSO) 11.07 (1H, br s), 8.44 (2H, d, J 6), 7.23 (2H, d, J 6), 7.16 (1H, d,J 8), 7.09 (1H, d, J 2), 7.01 (1H, td, J 8, 5.5), 6.72 (1H, dd, J 11.5, 8), 3.07 (2H, t, J 7), 2.96 (2H, t, J 7).

### Example 12

### 3-(2-(4-Pyridyl)ethyl-7-azaindole

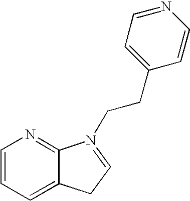

From 4-vinylpyridine and 7-azaindole; δ_{H} (400 MHz, d₆-DMSO) 8.40 (2H, d, J 6), 8.24 (1K d, J 4.5), 7.94 (1H, d, J 8), 7.47 (1H, d, J 3), 7.16 (2H, d, J 6), 7.07 (1H, dd, J 8, 4.5), 6.42 (1H, d, J 3), 4.55 (2H, t, J 7), 3.17 (2H, t, J 7).

### Example 13

### 13A. 5-Benzamidoindole

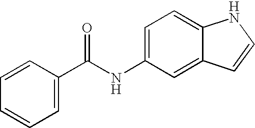

A mixture of 5-aminoindole (1.0 mmol), benzoic acid (1.0 mmol), 1-hydroxy-benzotriazole (1.1 mmol) and EDC hydrochloride (1.1 mmol) in DMF (5 ml) were stirred at room temperature until TLC analysis of the mixture showed the reaction to be complete. The solvent was removed under reduced pressure and the residue partitioned between water and ethyl acetate. The organic layer was dried (Na₂SO₄), filtered, evaporated and the residue purified by column chromatography on silica Elution with mixtures of petroleum ether and ethyl acetate afforded the title compound.

δ_{H} (400 M CD₃OD) 8.48 (2H, d, J 8), 8.39 (1H, s), 8.08 (3H, m), 7.91 (1H, d, J 8.5), 7.85 (1H, d, J 8.5), 7.18 (1H, d, J 3), 6.98 (1H, d, J 3).

### 13B. 5-Benzamido-3-(2-(pyrid-4-yl)ethyl)indole

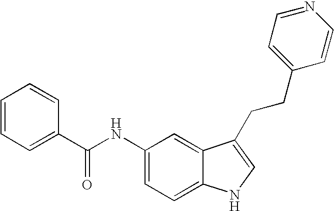

Reacting 4-vinylpyridine and 5-benzamidoindole under the conditions set out in Example 1. gave the title compound.

δ_{H} (400 MHz, d₆-DMSO) 10.76 (1H, br s), 10.10 (1H, br s), 8.44 (2H, d, J 6), 8.00 (2H, d, J 9), 7.98 (1H, s), 7.54 (3H, m), 7.42 (1H, dd, J 8.5, 1.5), 7.34 (1H, J 9), 7.27 (2H, d, J 6), 7.08 (1H, d, J2), 3.00 (4H, s).

### Example 14

### 14A. 5-(3-Fluoro-5-(4-morpholino)benzamido)indole

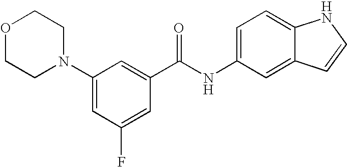

By following the methodology set out in Example 13A, but using 5-aminoindole and 3-fluoro-5-(4-morpholino)benzoic acid instead of 5-aminoindole and benzoic acid, the title compound was prepared.

δ_{H} (400 MHz, CDCl₃) 8.25 (1H, br s), 7.94 (1H, s), 7.81 (1H, br s), 7.38 (1H, d, J 8.5), 7.34 (1H, d, J 8.5), 7.24 (2H, m), 6.98 (1H, d, J 8.5), 6.72 (1H, dt, J 11.5, 2), 6.56 (1H, t, J 2), 3.86 (4H, m), 3.23 (4H, m).

### 14B. 5-(3-Fluoro-5-(4-morpholino)benzamido)-3-(2-(4-pyridyl)ethyl)indole

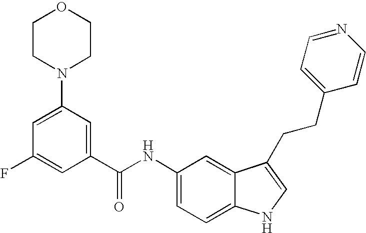

Reacting 4-vinylpyridine and 5-(3-fluoro-5-(4-morpholino)benzamido)indole under the conditions set out in Example 1 gave the title compound.

δ_{H} (400 MHz d₆-DMSO) 10.77 (1H, br s), 10.07 (1H, br s), 8.44 (2H, J 6), 7.96 (1H, d, J 2), 7.39 (1H, dd, J 9, 2), 7.36 (1H, t, J 2), 7.30 (1H, d, J 8.5), 7.27 (2H, d, J 6), 7.17 (1H, dm, J 9), 7.09 (1H, d, J 2), 6.98 (1H, dt, J 12, 2), 3.76 (4H, m), 3.24 (4H, m), 2.99 (4H, s).

### Example 15

### 15A. 6-(3-Fluoro-5-(4morpholino)benzamido)indole

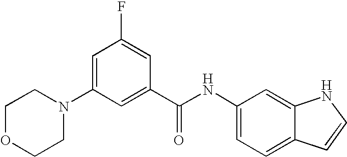

By following the methodology set out in Example 13A, but using 6-aminoindole and 3-fluoro-5-4-morpholino)benzoic acid instead of 5-aminoindole and benzoic acid, the intermediate title compound was prepared.

δ_{H} (400 MHz , d₆-DMSO) 11.07 (1H, br s), 10.11 (1H, br s), 8.02 (1H, s), 7.48 (1H, d, J 8.5), 7.32 (1H, s), 7.30 (1H, m), 7.25 (1H, dd, J 8.5, 2), 7.15 (1H, d, J 8.5), 6.98 (1H, dm, J 12.5), 6.38 (1H, s), 3.76 (4H, m), 3.24 (4H, m).

### 15B. 6-(3-Fluoro-5-(4-morpholino)benzamido)-3-(2-(4-pyridyl)ethyl)indole

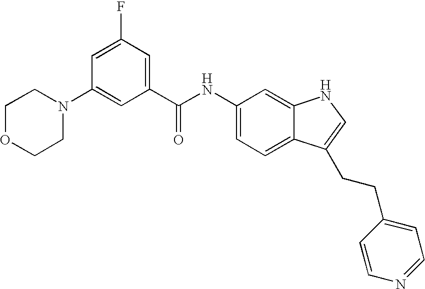

Reacting 4-vinylpyridine and 6-(3-fluoro-5-(4-morpholino)benzamido)indole under the conditions set out in Example 1 gave the title compound.

δ_{H} (400 MHz, d₆-DMSO) 10.78 (1H, br s), 10.10 (1H, br s), 8.44 (2H, d, J 6), 7.96 (1H, d, J 1.5), 7.50 (1H, d, J 8.5), 7.32 (1H, s), 7.28 (2H, d, J6), 7.24 (1H, dd, J 8.5, 1.5), 7.15 (1H, d, J 8.5), 7.06 (1H, d, J 2), 6.98 (1H, dm, J 12.5), 3.76 (4H, m), 3.24 (4H, m), 2.98 (4H, s).

### Example 16

### 16A. 5-(3-Trifluoromethoxybenzamido)indole

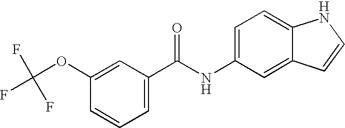

By following the methodology set out in Example 13A, but using 5-aminoindole and 3-(trifluoromethoxy)benzoic acid instead of 5-aminoindole and benzoic acid, the title compound was prepared.

δ_{H} (400 MHz , CDCl₃) 8.24 (1H, br s), 7.95 (1H, s), 7.85 (1H, s), 7.81 (1H, d, J 8), 7.77 (1H, s), 7.53 (1H, t, J 8), 7.37 (3H, m), 7.24 (1H, t, J 2.5), 6.55 (1H, s).

### 16B 5-(3-Trifluoromethoxybenzamido)-3-(2-(4-pyridy)ethyl)indole

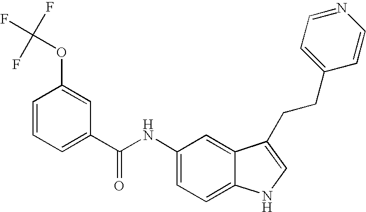

Reacting 4-vinylpyridine and 5-(3-trifluoromethoxybenzamido)indole under the conditions set out in Example 1 gave the title compound.

δ^{H} (400 MHz , CDCl₃) 8.46 (2H, d, J 6), 8.11 (1H, br s), 7.98 (2H, s), 7.83 (1H, d, J 8), 7.80 (1H, s), 7.54 (1H, t, J 8), 7.40 (1H, d, J 8), 7.32 (1H, t, J 8.5), 7.09 (2H, d, J 6), 6.89 (1H, d, J 2), 3.04 (4H, m).

### Example 17

### 17A. 5-(3-Trifluoromethylbenzamido)indole

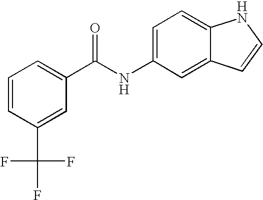

By following the methodology set out in Example 13A, but using 5-aminoindole and 3-(trifluoromethyl)benzoic acid instead of 5-aminoindole and benzoic acid, the title compound was prepared

δ_{H} (400 MHz, CDCl₃) 8.26 (1H, br s), 8.16 (1H, s), 8.10 (1H, d, J 7.5), 7.96 (1H, s), 7.92 (1H, s), 7.80 (1H, d, J 7.5), 7.63 (1H, t, J 7.5), 7.38 (2H, m), 7.24 (1H, m), 6.55 (1H, t, J 2).

### 17B. 5-(3-TrifluoromethylbenzaImido)-3-(2-(4-pyridylethyl)indole

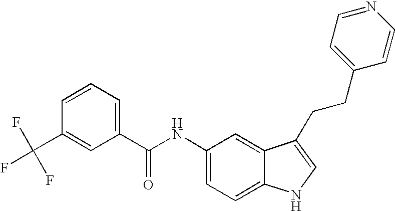

Reacting 4-vinylpyridine and 5-(3-trifluoromethylbenzamido)indole under the conditions set out in Example 1 gave the title compound.

δ_{H} (400 MHz, CDCl₃) 8.46 (2H, d, J 6), 8.18 (1H, s), 8.11 (1H, d, J 8), 8.06 (1H, s), 8.01 (1H, s), 7.98 (1H, s), 7.81 (1H, d, J 8), 7.64 (1H, t, J 8), 7.34 (2H, 7.10 (2H, d, J 6), 6.90 (1H, d, J 2), 3.06 (4H, m).

### Example 18

### 18A. 5-amino-1-(2-(4-pyridyl)ethyl)indole

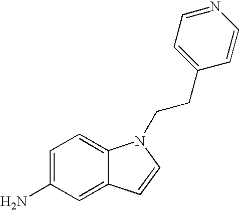

A mixture of 4-vinylpyridine (10.0 mmol), and 5-aminoindole (5.0 mmol), sodium (30 mg) and anhydrous copper sulphate (30 mg) in absolute ethanol (3 ml) were stirred at 130° C. in a sealed tube for 16 hours. Upon cooling to room temperature the solvent was removed under reduced pressure and the residue subjected to purification by flash chromatography on silica gel. Elution with 5% methanol in ethyl acetate afforded the title product.

δ_{H} (400 MHz , d₆-DMSO) 8.41 (2H, d, J 6), 7.18 (3H, m), 7.04 (1H, d, J 3), 6.65 (1H, d, J 2), 6.51 (1H, dd, J 8.5, 2), 6.06 (1H, d, J 3), 4.46 (2H, br s), 4.31 (2H, t, J 7), 3.04 (2H, t, J 7).

### 18B. 5-(3-Fluoro-5-(4-morpholino)benzamido)-1-(2-(4-pyridyl)ethyl)indole

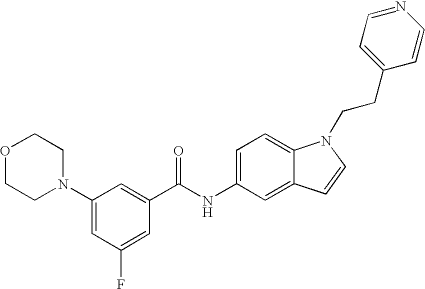

Reacting 5-amino-1-(2-(4-pyridyl)ethyl)indole (Example 18A) and 3-fluoro-5-(4-morpholino)-benzoic acid following the procedure set out in Example 13A gave the product shown above.

δ_{H} (400 MHZ, CD₃OD) 8.34 (2H, d, J 6), 7.87 (1H, s), 7.36 (3H, s), 7.14 (2H, d, J 6), 7.09 (1H, d, J 3), 6.92 (1H, dt, J 12, 2), 6.41 (1H, d, J 3), 4.52 (2H, t, J 70, 3.88 (4H, m), 3.26 (4H, m), 3.21 (2H, t, J 7).

### Example 19

### 1-(2-(4-pyridyl)ethyl)indole

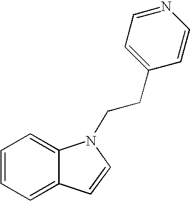

By following the procedure set out in Example 18A but substituting indole for 5-aminoindole, the product shown above was prepared.

δ_{H} (400 MHz d₆-DMSO) 8.41 (2H, d, J 6), 7.51 (2H, t, J 7.5), 7.27 (1H, d, J 3), 7.20 (2H, d, J 6), 7.11 (1H, tm, J 7.5), 6.99 (1H, tm, J 7.5), 6.37 (1H, d, J 3), 4.46 (2H, t, J 7.5), 3.09 (2H, t, J 7.5).

### Example 20

### 20A. 5-Amino-3-(2-(4-pyridyl)ethyl)indole

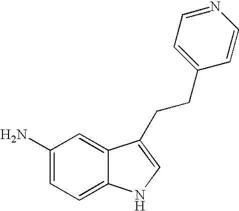

A mixture of 5-nitro-3-(2-(4-pyridyl)ethyl)indole (see Example 6) (0.5 mmol), iron powder (5.0 mmol), iron (II) sulphate heptahydrate (0.3 mmol) in 1,4-dioxane(6 ml) and water (1.5 ml) were stirred and at 90° C. for 2-3 hours. Upon cooling to room temperature the mixture was filtered, the solvent removed under reduced pressure and the residue subjected to purification by flash chromatography on silica gel. Elution with ethyl acetate or 10% methanol in ethyl acetate afforded the product shown. δ_{H} (400 MHz , d₆-DMSO) 10.25 (1H, br s), 8.44 (2H, d, J 6), 7.26 (2H, d, J 6), 7.02 (1H, d, J 8.5), 6.88 (1H, d, J 2), 6.70 (1H, d, J 2), 6.48 (1H, dd, J 8.5, 2), 4.42 (2H, br s), 2.91 (4H, m).

### 20B. 5-(Phenylcarbamoylamino)-3-(2-(4-pyridyl)ethyl)indole

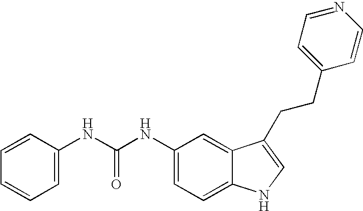

A solution of the 5-amino-3-(2-(4-pyridyl)ethyl)indole compound of Example 20A (0.1 mmol) in chloroform (1 ml) was stirred at 80° C. and treated with phenyl-isocyanate (0.1 mmol), stirred for 30 mins and cooled to room temperature. Filtration of the precipitate under reduced pressure afforded 5-(phenylcarbamoyl-amino)-3-(2-(4-pyridyl)ethyl)indole.

δ_{H} (400 MHz, d₆-DMSO) 10.65 (1H, br s), 8.56 (1H, br s), 8.44 (2H, d, J 6), 8.40 (1H, br s), 7.71 (1H, d, J 2), 7.46 (2H, d, J 7.5), 7.26 (5H, m), 7.04 (2H, m), 6.94 (1H, tt, J 7.5, 1), 2.98 (4H, m).

### Example 21

### 5-(Phenylcarbamoylamino)indole

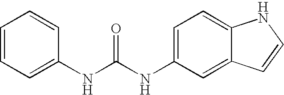

A solution of 5-aminoindole (0.1 mmol) in chloroform (1 ml) was stirred at 80° C. and treated with phenylisocyanate (0.1 mmol), stirred for 30 mins and cooled to room temperature. Filtration of the precipitate under reduced pressure afforded the intermediate product shown above.

δ_{H} (400 MHz, d₆-DMSO) 10.95 (1H, br s), 8.57 (1H, br s), 8.40 (1H, br s), 7.67 (1H, d, J 2), 7.46 (2H, d, J 7.5), 7.28 (4H, m), 7.06 (1H, dd, J 8.5, 2), 6.94 (1H, tt, J 7.5, 1), 6.35 (1H, m).

The compound of this example can be converted to 5-(phenylcarbamoylamino)-3-(2-(4-pyridyl)ethyl)indole by reaction with 4-vinylpyridine following the procedure of Example 1.

### Example 22

### 3-(2-(2-Chloro-4-pyrimidinyl)ethyl)indole

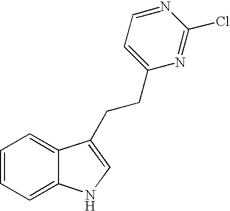

Reacting 2-chloro-4-vinylpyrimidine and indole under the conditions set out in Example 1 gave the title compound.

δ_{H} (400 MHz, d₆-DMSO) 10.79 (1H, br s), 8.61 (1H, d, J 5), 7.53 (1H, d, J 8), 7.47 (1H, d, J 5), 7.32 (1H, d, J 8), 7.11 (1H, d, J 2), 7.06 (1H, t, J 8), 6.97 (1H, t, J 8), 3.12 (4H, m).

### Example 23

### 23A. 5-(2-(4-Morpholino)isonicotinamido))indole

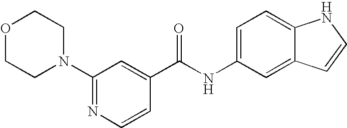

5-Aminoindole and 2-(4-morpholino)isonicotinic acid were reacted together under the conditions set out in Example 13A to give the title compound.

δ_{H} (400 MHz, d₆-DMSO) 11.07 (1H, br s), 10.17 (1H, br s), 8.28 (1H, d, J 5), 7.96 (1H, s), 7.36 (2H, s), 7.34 (1H, t, J 3), 7.28 (1H, s), 7.15 (1H, dd, J 5, 2), 6.42 (1H, dd, J 3, 2), 3.73 (4H, t, J 5), 3.53 (4H, t, J 5).

### 23B. 5-(2-(4-Morpholino)isonicotinamido)-3-(2-(4-pyridyl)ethyl)indole

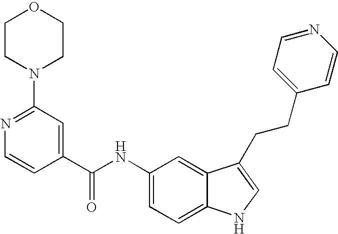

4-Vinylpyridine and 5-(2-(4-morpholino)isonicotinamido)indole were reacted together under the conditions described in Example 1 to give the title compound.

δ_{H} (400 MHz, d₆-DMSO) 10.78 (1H, br s), 10.19 (1H, br s), 8.44 (2H, d, J 6), 8.28 (1H, d, J 5), 7.97 (1H, br s), 7.39 (1H, dd, J 8.5, 2), 7.29 (4H, m), 7.16 (1H, dd, J 5, 1), 7.10 (1H, s), 3.73 (4H, t, J 5), 3.53 (4H, t, J 5), 3.00 (4H, s).

### Example 24

### 24A. 5-(1-phthalimido) indole

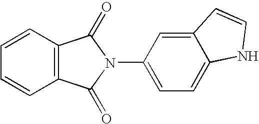

A mixture of 5-aminoindole (3.0 mmol) and phthalic anhydride (3.0 mmol) in toluene (5 ml) was stirred and held at reflux temperature for 2-3 hours. Upon cooling to room temperature the solvent was removed under reduced pressure and the residue purified by column chromatography on silica. Elution with diethyl ether afforded the title compound.

δ_{H} (400 MHz, d₆-DMSO) 11.32 (1H, br s), 7.96 (2H, m), 7.90 (2H, m), 7.57 (1H, d, J 2), 7.50 (1H, d, J 8.5), 7.46 (1H, t, J 2), 7.09 (1H, dd, J 8.5, 2), 6.51 (1H, m).

### 24B. 5-(1-Phthalimido)-3-(2-(4-pyridyl)ethyl)indole

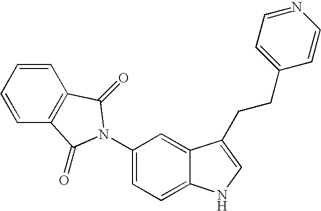

4-Vinylpyridine and 5-(1-phthalimido)indole were reacted together under the conditions set out in Example 1 to give the title compound.

δ_{H} (400 MHz, d₆-DMSO) 11.03 (1H, br s), 8.43 (2H, d, J 6), 7.97 (2H, m), 7.91 (2H, m), 7.64 (1H, d, J 2), 7.44 (1H, d, J 8.5), 7.28 (2H, d, J 6), 7.23 (1H, d, J 2), 7.08 (1H, dd, J 8.5, 2), 3.00 (4H, m).

### Example 25

### 25A. 6-Amino-1-(2-(4-pyridyl)ethyl)indole

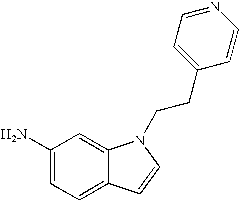

4-Vinylpyridine and 6-aminoindole were reacted together according to the method of Example 18A to give the title compound.

δ_{H} (400 MHz, d₆-DMSO) 8.44 (2H, d, J 6), 7.21 (2H, d, J 6), 7.17 (1H, d, J 8), 6.88 (1H, d, J 3), 6.60 (1H, d, J 2), 6.41 (1H, dd, J 8, 2), 6.13 (1H, d, J 3), 4.77 (2H, br s), 4.24 (2H, t, J 7.5), 3.04 (2H, t, J 7.5).

### 25B. 6-(3-Fluoro-5-(4-morpholino)benzamido)-1-(2-(4-pyridyl)ethyl)indole

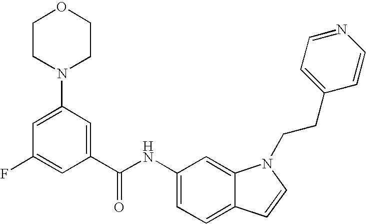

Using the method set out in Example 13A, 6-amino-1-(2-(4-pyridyl)ethyl)indole and 3-fluoro-5-(4-morpholino)benzoic acid were reacted together to give the title compound.

δ_{H} (400 MHz, d₆-DMSO) 10.24 (1H, br s), 8.50 (2H, d, J 6), 8.11 (1H, br s), 7.55 (1H, d, J 8.5), 7.42 (1H, br s), 7.37 (1H, dd, J 8.5, 1.5), 7.26 (4H, m), 7.06 (1H, dt, J 12.5, 2), 6.40 (1H, d, J 3), 4.48 (2H, t, J 7), 3.83 (4H, t, J 5), 3.31 (4H, t, J 5), 3.18 (2H, t, J 7).

### Example 26

### 5-(4-(2-oxo-pyrrolidin-1-yl)benzamido)-3-(2-(4-pyridyl)ethyl)indole

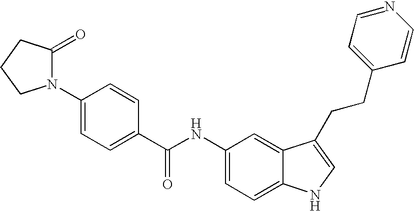

5-Amino-3-(2-(4-pyridyl)ethyl)indole and 4-(2-oxo-pyrrolidin-1-yl)benzoic acid were reacted together under the conditions set out in Example 13A to give the title compound.

δ_{H} (400 MHz, d₆-DMSO) 10.74 (1H, br s), 10.03 (1H, br s), 8.44 (2H, d, J 6), 8.02 (2H, d, J 9), 8.00 (1H, s), 7.82 (2H, d, J 9), 7.42 (1H, d, J 8.5), 7.30 (1H, d, J 8.5), 7.28 (2H, d, J 6), 7.09 (1H, s), 3.90 (2H, t, J 7.5), 3.00 (4H, s), 2.55 (2H, t, J 7.5), 2.09 (2H, quin, J 7.5).

### Example 27

### 3-(2-(2-(2-Hydroxyethylamino -4-pyrimidinyl)ethyl)indole

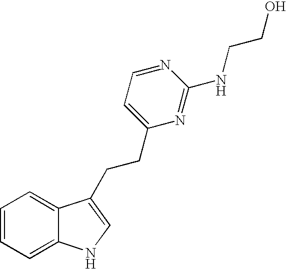

A mixture of 3-(2-(2-chloro-4-pyrimidinyl)ethyl)indole (0.5 mmol) and ethanolamine (0.5 ml) was stirred and irradiated in a microwave at 250° C. for 5 minutes. Upon cooling to room temperature water was added, the organics were extracted into ethyl acetate, dried (Na₂SO₄), filtered and evaporated and the resulting residue was subjected to column chromatography on silica. Elution with diethyl ether or ethyl acetate afforded the title product.

δ_{H} (400 MHz , d₆-DMSO) 10.76 (1H, br s), 8.12 (1H, d J 5), 7.52 (1H, d, J 8), 7.32 (1H, d, J 8), 7.11 (1H, s), 7.06 (1H, t, J 8), 6.97 (1H, t, J 8), 6.90 (1H, br s), 6.50 (1H, d, J 5), 4.68 (1H, br s), 3.51 (2H, t, J 6), 3.34 (2H, t, J 6), 3.04 (2H, t, J 8), 2.87 (2H, t, J 8).

### Example 28

### 3-(2-(2-(3-Hydroxy-2-methyl-prop-2-ylamino)-4-pyrimidinyl)ethyl)indole

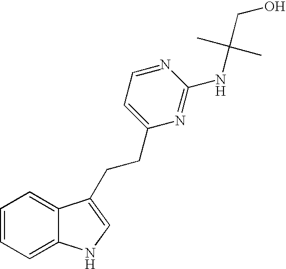

A mixture of 3-(2-(2-chloro-4-pyrimidinyl)ethyl)indole (0.5 mmol) and 2-amino-2-methylpropan-1-ol (0.5 ml) was stirred and irradiated in a microwave at 200° C. for 5 minutes. The reaction mixture was allowed to cool and was worked up as described in Example 27 to give the title compound.

δ_{H} (400 MHz, d₆-DMSO) 10.76 (1H, br s), 8.11 (1H, d, J 5), 7.51 (1H, d, J 8), 7.32 (1H, d, J 8), 7.10 (1H, s), 7.06 (1H, t, J 8), 6.96 (1H, t, J 8), 6.50 (1H, d, J 5), 6.31 (1H, br s), 5.11 (1H, t, J 5.5), 3.49 (2H, d, J 5.5),3.05 (2H, t, J 7.5),2.86 (2H, t, J 7.5), 1.32 (6H, s).

### Example 29

### 3-(2-(2-((S)-(−)-α-methylbenzylamino)-4-pyrimidinyl)ethyl)indole

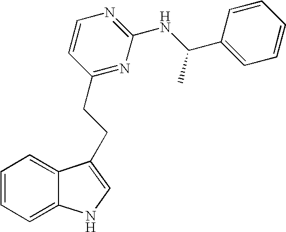

A mixture of 3-(2-(2-chloro-4-pyrimidinyl)ethyl)indole (0.5 mmol) and (S)-(−)-α-methylbenzylamine (0.5 ml) was stirred and irradiated in a microwave at 200° C. for 5 minutes. The mixture was worked up according to the method of Example 27 to give the title compound.

δ_{H} (400 MHz, d₆-DMSO) 10.73 (1H, br s), 8.07 (1H, d, J 5), 7.58 (1H, d, J 7.5), 7.49 (1H, d, J 7.5), 7.40 (2H, d, J 7.5), 7.30 (3H, m), 7.17 (1H, t, J 7.5), 7.05 (2H, t, J 7.5), 6.96 (1H, t, J 7.5), 6.44 (1H, br s), 5.11 (1H, m, J 7), 3.00 (2H, br s), 2.83 (2H, t, J 7.5), 1.43 (3H, d, J 7).

### Example 30

### 3-(2-(2-((R)-(+)-α-methylbenzylamino)-4-pyrimidinyl)ethyl)idole

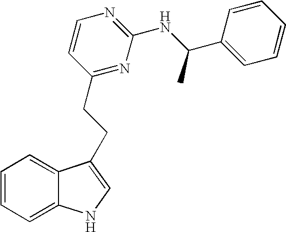

3-(2-(2-Chloro-4-pyrimidinyl)ethyl)indole and (R)-(+)-α-methylbenzylamine were reacted together according to the method of Example 29 to give the title compound.

δ_{H} (400 MHz, d₆-DMSO) 10.73 (1H, br s), 8.07 (1H, d, J 5), 7.58 (1H, d, J 7.5), 7.49 (1H, d, J 7.5), 7.40 (2H, d, J 7.5), 7.30 (3H, m), 7.17 (1H, t, J 7.5), 7.05 (2H, t, J 7.5), 6.96 (1H, t, J 7.5), 6.44 (1H, br s), 5.11 (1H, m, J 7), 3.00 (2H, br s), 2.83 (2H, t, J 7.5), 1.43 (3H, d, J 7).

### Example 31

### 3-(2-(4-Pyrimidinyl)ethyl)indole

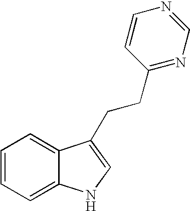

A mixture of 3-(2-(2-chloro-4-pyrimidinyl)ethyl)indole (0.35 mmol), 10% palladium on carbon (20 mg) and triethylamine (0.7 mmol) in ethanol (2 ml) was stirred at room temperature under an atmosphere of hydrogen for 16 hours. The mixture was filtered, evaporated and the resulting residue subjected to column chromatography on silica. Elution with diethyl ether afforded the title compound.

δ_{H} (400 MHz, d₆-DMSO) 10.77 (1H, br s), 9.10 (1H, d, J 1), 8.63 (1H, d, J 5.5), 7.52 (1H, d, J38), 7.42 (1H, dd, J 5, 1), 7.32 (1H, d, J 8),7.09 (1H, s), 7.06 (1H, t, J 8), 6.97 (1H, t, J 8), 3.11 (4H, s).

### Example 32

### 5-(3-tert-Butyl-1-phenylpyrazol-5-ylcarbamoylamino)-3-(2-(4-pyridyl)ethyl)indole

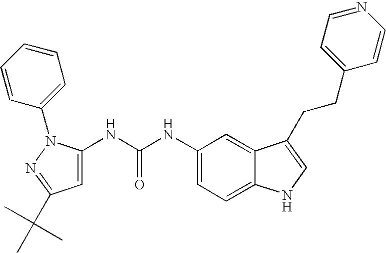

A mixture of 5-amino-3-tert-butyl-1-phenylpyrazole (0.5 mmol), and carbonyldiimidazole (0.55 mmol) in dichloromethane (2 ml) was stirred at room temperature for 6-8 hours. 5-Amino-3-(2-(4-pyridyl)ethyl)indole (0.5 mmol) was added and the mixture was stirred and held at reflux overnight. The mixture was then cooled to room temperature, the solvent evaporated and the resulting residue subjected to column chromatography on silica Elution with ethyl acetate afforded the title compound.

δ_{H} (400 MHz, d₆-DMSO) 10.66 (1H, br s), 8.78 (1H, br s), 8.43 (2H d, J 6), 8.29 (1H, br s), 7.68 (1H, d, J 2), 7.54 (4H, d, J4), 7.42 (1H, m), 7.26 (2H, d, J 6), 7.22 (11H, d, J 8.5), 7.04 (1H, d, J 2), 6.98 (1H, dd, J 8.5, 2), 6.38 (1H, s), 2.69 (4H, s), 1.28 (9H, s).

### Example 33

### E-1[(4-Methylphenyl)sulphonyl]-3-(2-(3-pyridyl)ethenyl)indole

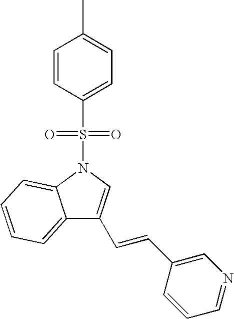

A stirred solution of triphenyl(3-pyridylmethyl)phosphonium chloride hydrochloride (3.0 mmol) in anhydrous tetrahydrofuran (30 ml) under a nitrogen atmosphere was cooled to −78° C. and treated with n-butyllithium (1.6 M in hexane, 4.0 ml, 6.4 mmol) dropwise over 10 minutes. The resulting solution was stirred at −78° C. for a further 30 minutes, 1-[(4-methylphenyl)sulphonyl]indole-3-carbaldehyde was added (3.0 mmol) and the mixture stirred at room temperature overnight. Water was added, the mixture extracted with diethyl ether, the organic layer dried (Na₂SO₄), filtered and evaporated and the resulting residue subjected to column chromatography on silica. Elution with mixtures of petroleum ether and ethyl acetate afforded the title compound.

δ_{H} (400 MHz, d₆-DMSO) 8.43 (2H, m), 7.91 (1H, d, J 8), 7.78 (2H, d, J 8), 7.54 (2H, m), 7.39 (2H, d, J 8), 7.34 (1H, t, J 8), 7.26 (2H, m), 7.17 (1H, t, J 8), 6.82 (2H, s), 2.33 (3H, s).

### Example 34

### 1-[(4-Methylphenyl)sulphonyl]-3-(2-(3-pridyl)ethyl)indole

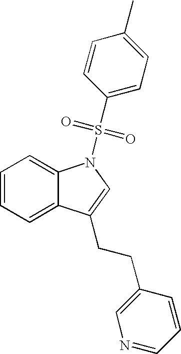

A mixture of E-1-[(4-methylphenyl)sulphonyl]-3-(2-(3-pyridyl)ethenyl)indole (0.5 mmol) and 10% palladium on carbon (20 mg) in ethanol (2 ml) was stirred at room temperature under an atmosphere of hydrogen for 16 hours. The mixture was filtered, evaporated and the resulting residue subjected to column chromatography on silica. Elution with mixtures of petroleum ether and ethyl acetate afforded the title compound.

δ_{H} (400 MHz, d₆-DMSO) 8.43 (1H, d, J 1.5), 8.40 (1H, dd, J 4.5, 1.5), 7.87 (1H, d, J 8.5), 7.72 (2H, d, J 8.5), 7.62 (2H, m), 7.51 (1H, s), 7.33 (3H, m), 7.26 (2H, m), 2.98 (4H, s), 2.31 (3H, s).

### Example 35

### 3-(2-(3-pyridyl)ethyl)indole

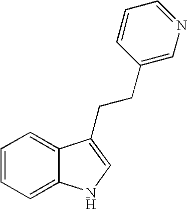

A mixture of 1-[(4-methylphenyl)sulphonyl]-3-(2-(3-pyridyl)ethyl)indole (0.5 mmol) and 2M potassium hydroxide (0.5 ml) in methanol (2 ml) was stirred and held at reflux for 40 hours whereupon the mixture was cooled to room temperature and evaporated. Water was added, the mixture extracted with ethyl acetate, the organic layer dried (Na₂SO₄), filtered and evaporated and the resulting residue subjected to column chromatography on silica. Elution with diethyl ether afforded the title compound.

δ_{H} (400 MHz, d₆-DMSO) 10.77 (1H, br s), 8.43 (1H, d, J 1.5), 8.38 (1H, dd, J 5, 1.5), 7.66 (1H, dt, J8, 1.5), 7.54 (1H, d, J8), 7.32 (lH, d, J 8), 7.28 (1H, d, J 8, 5), 7.08 (1H, s), 7.06 (1H, t, J 8), 6.97 (1H, t, J 8), 2.98 (4H, s).

Biological Activity

### Example 36

p38 MAP Kinase Inhibitory Activity

Compounds of the invention were tested for p38 MAP kinase inhibitory activity using the following protocol.

In 1 ml of fresh assay buffer (20 mM BEPES pH 7.4, 25 mM β-glycerophosphate, 5 mM EDTA, 15 mM MgCl₂, 100 μM ATP, 1 mM sodium orthovanadate, 1 mM DT, 35 μg of inactive purified α p38 and 0.1 2 μg of active MKK6 (1688 U/mg—Upstate Biotechnology) were mixed and incubated at room temperature overnight to activate the p38. The activated p38 was then diluted six-fold with assay buffer, and 10 μl mixed with 10 μl of MBP mix (150 μl 10× strength assay buffer (250 mM HEPES pH 7.4, 250 mM β-glycerophosphate, 50 mM EDTA, 150 mM MgCl₂), 1.5 μM of 10 mM DDT and 10 mM sodium orthovanadate, 7.5 μM of 10 mM ATP, 723 μM water, 35 μCiγ³³ P-ATP, 100 μl myelin basic protein (MBP) (5 mg/ml)) and added to 96 well plates along with 5 μl of various dilutions of the test compound in DMSO (up to 10%). The reaction was allowed to proceed for fifty minutes before being stopped with an excess of ortho-phosphoric acid (30 μl at 2%). γ³³ P-ATP which remained unincorporated into the myelin basic protein was separated from phoshorylated MBP on a Mlipore (RTM) MAPH filter plate. The wells of the MAPH plate were wetted with 0.5% orthophosphoric acid, and then the results of the reaction were filtered with a Millipore vacuum filtration unit through the wells. Following filtration, the residue was washed twice with 200 μl of 0.5% orthophosphoric acid. Once the filters had dried, 25μ of Microscint 20™ scintillant was added, and then counted on a Packard Topcount™ counter for 30 seconds. The percentage inhibition of the p38 activity was calculated and plotted in order to determine the concentration of the test compound required to inhibit 50% of the p38 activity (IC₅₀). The results are shown in Table 1 below.

TABLE 1

| Compound of | p38 Activity Data - IC₅₀ Values |
|---|---|
| Example Number | (μM unless stated) |
| 1 | 33 |
| 2 | 240 |
| 3 | 820 |
| 4 | 250 |
| 5 | 122 |
| 6 | 300 |
| 7 | 650 |
| 13B | 40 |
| 8 | 270 |
| 14B | 500 nM |
| 9 | 111 |
| 10 | 35 |
| 16B | 2 |
| 17B | 4 |
| 18B | 3 |
| 19 | 200 |
| 20A | 23% @ 1 mM |
| 20B | 28 |
| 11 | 45 |
| 12 | 177 |
| 15B | 36% @ 3 μM |
| 22 | 120 |
| 23B | 1.3 |
| 24B | 25% @ 300 μM |
| 25A | 475 |
| 25B | 300 nM |
| 26 | 28% @ 1 μM |
| 27 | 38 |
| 28 | 1.5 |
| 29 | 3 |
| 30 | 15 |
| 31 | 715 |
| 32 | 145 nM |
| 33 | 1 mM |
| 34 | >1 mM |
| 35 | 1 mM |

### Example 37

Inhibition of LPS-Induced TNF-α Production in THP-1 Cells, In Vitro Assay

The ability of the compounds of this invention to inhibit the TNF-α release was determined using a rninor modification of the methods described in Rawlins P., et al., “Inhibition of endotoxin-induced TNF-α production in macrophages by 5Z-7-oxo-zeaenol and other fungal resorcyclic acid lactones,” International J. of Immunopharmacology, 21, 799, (1999).

THP-1 cells, human monocytic leukaemic cell line, ECACC) were maintained in culture medium [RPMI 1640 (Invitrogen) and 2 mM L-Glutamine supplemented with 10% foetal bovine serum (Invitrogen)] at approximately 37° C. in humidified 5% CO₂ in stationary culture. TBP-1 cells were suspended in culture medium containing 50 ng/ml PMA (SIGMA), seeded into a 96-well tissue culture plate (IWAKI) at 1×10⁵ cells/well (100 μl/well) and incubated as described above for approximately 48 h. The medium was then aspirated, the wells washed twice in Phosphate Buffered Saline and 1 μg/ml LPS (SIGMA) in culture medium was added (200 μl/well).

Test compounds were reconstituted in DMSO (SIGMA) and then diluted with the culture medium such that the final DMSO concentration was 0.1%. Twenty microlitre aliquots of test solution or medium only with DMSO (solvent control) were added to triplicate wells immediately following LPS addition, and incubated for 6 h as described above. Culture supernatants were collected and the amount of human TNF-α present was determined by ELISA (R&D Systems) performed according to the manufacturer's instructions.

The IC₅₀ was defined as the concentration of the test compound corresponding to half maximal inhibition of the control activity by non-linear regression analysis of their inhibition curves. The IC₅₀ for the compound Of 14B (5-(3-Fluoro-5-(4-morpholino)benzamido)-3-(2-(4-pyridyl)ethyl)indole) was found to be 530 nM.

Pharmaceutical Formulations

### Example 38

(i) Tablet Formulation

A tablet composition containing a compound of the formula (I) is prepared by mixing 50 mg of the compound with 197 mg of lactose (BP) as diluent, and 3 mg magnesium stearate as a lubricant and compressing to form a tablet in known manner.

(ii) Capsule Formulation

A capsule formulation is prepared by mixing 100 mg of a compound of the formula (I) with 100 mg lactose and filling the resulting mixture into standard opaque hard gelatin capsules.

Equivalents

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1-67. (canceled)

68. A method for the prophylaxis or treatment of a disease state or condition mediated by a p38 MAP kinase, which method comprises administering to a subject in need thereof a compound of the formula (I): 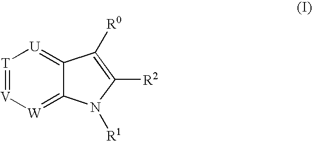
or a salt thereof;
wherein U, T, V and W are each a nitrogen atom or a group CR⁴ provided that no more than three of U, T, V and W are nitrogen atoms;
R⁰ is hydrogen, C₁₋₄ hydrocarbyl, halogen or a group -A-R³;
R¹ is hydrogen, C₁₋₄ hydrocarbyl or a group -A-R³; provided that only one of R⁰ and R¹ is a group -A-R³;
R² is hydrogen, C₁₋₄ hydrocarbyl or halogen;
A is a carbon- or heteroatom-containing linker group having a linking chain length of one or two atoms;
R³ is a monocyclic or bicyclic heteroaryl group containing from five to twelve ring members;
each group R⁴ is independently selected from hydrogen, hydroxy, halogen, nitro, cyano, a monocyclic heterocyclic group having up to seven ring members, a group N(R⁵)₂, a group C(O)N(R₆)₂, a group SO₂N(R⁶)₂, a group R^{a}—R^{b} and a group Y; provided that no more than one group Y is present;
R^{a} is a bond, O, S, SO, SO₂, NH or N—C₁₋₄ hydrocarbyl;
R^{b} is C₁₋₈ hydrocarbyl optionally interrupted by O, S, SO, SO₂, NH or N—C₁₋₄ hydrocarbyl and optionally substituted by one or more substituents selected from hydroxy, amino, mono- or di-C₁₋₄ hydrocarbylamino, C₁₋₄ hydrocarbyloxy, oxo, C ₁₋₄ hydrocarbylthio and halogen;
each group R⁵ is independently selected from hydrogen, C₁₋₄ alkyl, C₁₋₄ acyl and C₁₋₄ alkylsulphonyl;
each group R₆ is independently selected from hydrogen and C₁₋₄ hydrocarbyl;
Y is a group —N(R⁷)—C(O)—R⁸ or —N(R⁷)—SO₂—R⁸;
R⁷ is hydrogen, C₁₋₄ hydrocarbyl or a group C(O)—R⁸ or SO₂—R⁸;
R⁸ is selected from C₁₋₁₀ hydrocarbyl, C₁₋₁₀ hydrocarbylamino, C₁₋₁₀ hydrocarbylthio, C₁₋₁₀ hydrocarbyloxy, and aryl, arylamino, arylthio and aryloxy groups, the aryl moieties of which are carbocyclic or heterocyclic and have from five to twelve ring members, each substituent group R⁸ being optionally substituted by one or more groups R⁴ other than Y; or R⁷ and R⁸ together with the nitrogen and carbon or sulphur atoms to which they are attached are linked to form a ring structure of 4 to 7 ring members;
wherein R⁰ is other than a 2-(2,4-diamino-6-triazinyl)ethyl group when, in combination, U, T, V and W are all CH, and R¹ and R² are both hydrogen;
and provided that when the group -A-R³ contains an acidic substitituent group selected from carboxylic, phosphonic and sulphonic acids and tetrazoles, or contains a —C(O)NSO₂— group, or when -A- is —C(O)N— and the nitrogen atom of the group A is linked directly to a furan or thiophene ring, then either R¹ is -A-R³ and both R⁰ and R² are hydrogen, or R⁰ is -A-R³ and R¹ is hydrogen.

69. A method according to claim 68 wherein the disease state or condition is selected from rheumatoid arthritis, osteoarthritis, rheumatoid spondylitis, gouty arthritis, traumatic arthritis, rubella arthritis, psoriatic arthritis, and other arthritic conditions; Alzheimer's disease; toxic shock syndrome, the inflammatory reaction induced by endotoxin or inflammatory bowel disease; tuberculosis, atherosclerosis, muscle degeneration, Reiter's syndrome, gout, acute synovitis, sepsis, septic shock, endotoxic shock, gram negative sepsis, adult respiratory distress syndrome, cerebral malaria, chronic pulmonary inflammatory disease, silicosis, pulmonary sarcoisosis, bone resorption diseases, reperfusion injury, graft vs. host reaction, allograft rejections, fever and myalgias due to infection, cachexia, cachexia secondary to infection or malignancy, cachexia secondary to acquired immune deficiency syndrome (AIDS), AIDS, ARC (AIDS related complex), keloid formation, scar tissue formation, Crohn's disease, ulcerative colitis, pyresis, chronic obstructive pulmonary disease (COPD), acute respiratory distress syndrome (ARDS), asthma, pulmonary fibrosis, bacterial pneumonia, proliferative diseases, such as cancers (particular colon and breast cancer) and alopecia.

70. A method for the prophylaxis or treatment of a disease state or condition selected from rheumatoid arthritis, osteoarthritis, rheumatoid spondylitis, gouty arthritis, traumatic arthritis, rubella arthritis, psoriatic arthritis, and other arthritic conditions; Alzheimer's disease; toxic shock syndrome, the inflammatory reaction induced by endotoxin or inflammatory bowel disease; tuberculosis, atherosclerosis, muscle degeneration, Reiter's syndrome, gout, acute synovitis, sepsis, septic shock, endotoxic shock, gram negative sepsis, adult respiratory distress syndrome, cerebral malaria, chronic pulmonary inflammatory disease, silicosis, pulmonary sarcoisosis, bone resorption diseases, reperfusion injury, graft vs. host reaction, allograft rejections, fever and myalgias due to infection, cachexia, cachexia secondary to infection or malignancy, cachexia secondary to acquired immune deficiency syndrome (AIDS), AIDS, ARC (AIDS related complex), keloid formation, scar tissue formation, Crohn's disease, ulcerative colitis, pyresis, chronic obstructive pulmonary disease (COPD), acute respiratory distress syndrome (ARDS), asthma, pulmonary fibrosis, bacterial pneumonia, proliferative diseases, such as cancers (particular colon and breast cancer) and alopecia; which method comprises administering to a subject in need thereof a compound of the formula (I): 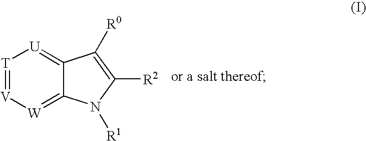
wherein U, T, V and W are each a nitrogen atom or a group CR⁴ provided that no more than three of U, T, V and W are nitrogen atoms;
R⁰ is hydrogen, C₁₋₄ hydrocarbyl, halogen or a group -A-R³;
R¹ is hydrogen, C₁₋₄ hydrocarbyl or a group -A-R³; provided that only one of R⁰ and R¹ is a group -A-R³;
R² is hydrogen, C₁₋₄ hydrocarbyl or halogen;
A is a carbon- or heteroatom-containing linker group having a linking chain length of one or two atoms;
R³ is a monocyclic or bicyclic heteroaryl group containing from five to twelve ring members;
each group R⁴ is independently selected from hydrogen, hydroxy, halogen, nitro, cyano, a monocyclic heterocyclic group having up to seven ring members, a group N(R⁵)₂, a group C(O)N(R⁶)₂, a group SO₂N(R⁶)₂, a group R^{a}—R^{b} and a group Y; provided that no more than one group Y is present;
R^{a} is a bond, O, S, SO, SO₂, NH or N—C₁₋₄ hydrocarbyl;
R^{b} is C₁₋₈ hydrocarbyl optionally interrupted by O, S, SO, SO₂, NH or N—C₁₋₄ hydrocarbyl and optionally substituted by one or more substituents selected from hydroxy, amino, mono- or di-C₁₋₄ hydrocarbylamino, C₁₋₄ hydrocarbyloxy, oxo, C₁₋₄ hydrocarbylthio and halogen;
each group R⁵ is independently selected from hydrogen, C₁₋₄ alkyl, C₁₋₄ acyl and C₁₋₄ alkylsulphonyl;
each group R⁶ is independently selected from hydrogen and C₁₋₄ hydrocarbyl;
Y is a group —N(R⁷)—C(O)—R⁸ or —N(R⁷)—SO₂—R⁸;
R⁷ is hydrogen, C₁₋₄ hydrocarbyl or a group C(O)—R⁸ or SO₂—R⁸;
R⁸ is selected from C₁₋₁₀ hydrocarbyl, C₁₋₁₀ hydrocarbylamino, C₁₋₁₀ hydrocarbylthio, C₁₋₁₀ hydrocarbyloxy, and aryl, arylamino, arylthio and aryloxy groups, the aryl moieties of which are carbocyclic or heterocyclic and have from five to twelve ring members, each substituent group R⁸ being optionally substituted by one or more groups R⁴ other than Y; or R⁷ and R⁸ together with the nitrogen and carbon or sulphur atoms to which they are attached are linked to form a ring structure of 4 to 7 ring members;
wherein R⁰ is other than a 2-(2,4-diamino-6-triazinyl)ethyl group when, in combination, U, T, V and W are all CH, and R¹ and R² are both hydrogen;
and provided that when the group -A-R³ contains an acidic substitituent group selected from carboxylic, phosphonic and sulphonic acids and tetrazoles, or contains a —C(O)NSO₂— group, or when -A- is —C(O)N— and the nitrogen atom of the group A is linked directly to a furan or thiophene ring, then either R¹ is -A-R³ and both R⁰ and R² are hydrogen, or R⁰ is -A-R³ and R¹ is hydrogen.

71. A method according to claim 69 wherein the disease state or condition is selected from inflammatory diseases and conditions, rheumatoid arthritis and osteoarthritis.

72. A method according to claim 70 wherein the disease state or condition is selected from inflammatory diseases and conditions, rheumatoid arthritis and osteoarthritis.

73. A method of inhibiting a p38 MAP kinase, which method comprises contacting the p38 MAP kinase with a kinase-inhibiting compound of the formula (I): 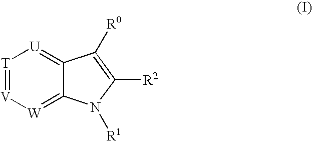
or a salt thereof;
wherein U, T, V and W are each a nitrogen atom or a group CR⁴ provided that no more than three of U, T, V and W are nitrogen atoms;
R⁰ is hydrogen, C₁₋₄ hydrocarbyl, halogen or a group -A-R³;
R¹ is hydrogen, C₁₋₄ hydrocarbyl or a group -A-R³; provided that only one of R⁰ and R¹ is a group -A-R³;
R² is hydrogen, C₁₋₄ hydrocarbyl or halogen;
A is a carbon- or heteroatom-containing linker group having a linking chain length of one or two atoms;
R³ is a monocyclic or bicyclic heteroaryl group containing from five to twelve ring members;
each group R⁴ is independently selected from hydrogen, hydroxy, halogen, nitro, cyano, a monocyclic heterocyclic group having up to seven ring members, a group N(R⁵)₂, a group C(O)N(R⁶)₂, a group SO₂N(R⁶)₂, a group R^{a}—R^{b} and a group Y; provided that no more than one group Y is present;
R^{a} is a bond, O, S, SO, SO₂, NH or N—C₁₋₄ hydrocarbyl;
R^{b} is C₁₋₈ hydrocarbyl optionally interrupted by O, S, SO, SO₂, NH or N—C₁₋₄ hydrocarbyl and optionally substituted by one or more substituents selected from hydroxy, amino, mono- or di-C₁₋₄ hydrocarbylamino, C₁₋₄ hydrocarbyloxy, oxo, C₁₋₄ hydrocarbylthio and halogen;
each group R⁵ is independently selected from hydrogen, C₁₋₄ alkyl, C₁₋₄ acyl and C₁₋₄ alkylsulphonyl;
each group R⁶ is independently selected from hydrogen and C₁₋₄ hydrocarbyl;
Y is a group —N(R⁷)—C(O)—R⁸ or —N(R⁷)—SO₂—R⁸;
R⁷ is hydrogen, C₁₋₄ hydrocarbyl or a group C(O)—R⁸ or SO₂—R⁸;
R⁸ is selected from C₁₋₁₀ hydrocarbyl, C₁₋₁₀ hydrocarbylamino, C₁₋₁₀ hydrocarbylthio, C₁₋₁₀ hydrocarbyloxy, and aryl, arylamino, arylthio and aryloxy groups, the aryl moieties of which are carbocyclic or heterocyclic and have from five to twelve ring members, each substituent group R⁸ being optionally substituted by one or more groups R⁴ other than Y; or R⁷ and R⁸ together with the nitrogen and carbon or sulphur atoms to which they are attached are linked to form a ring structure of 4 to 7 ring members;
wherein R⁰ is other than a 2-(2,4-diamino-6-triazinyl)ethyl group when, in combination, U, T, V and W are all CH, and R¹ and R² are both hydrogen;
and provided that when the group -A-R³ contains an acidic substitituent group selected from carboxylic, phosphonic and sulphonic acids and tetrazoles, or contains a —C(O)NSO₂— group, or when -A- is —C(O)N— and the nitrogen atom of the group A is linked directly to a furan or thiophene ring, then either R¹ is -A-R³ and both R^{o} and R² are hydrogen, or R⁰ is -A-R³ and R¹ is hydrogen.

74. A method of modulating a cellular process by inhibiting the activity of a p38 MAP kinase using a compound of the formula (I): 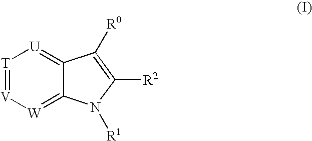
or a salt thereof;
wherein U, T, V and W are each a nitrogen atom or a group CR⁴ provided that no more than three of U, T, V and W are nitrogen atoms;
R⁰ is hydrogen, C₁₋₄ hydrocarbyl, halogen or a group -A-R³;
R¹ is hydrogen, C₁₋₄ hydrocarbyl or a group -A-R³; provided that only one of R⁰ and R¹ is a group -A-R³;
R₂ is hydrogen, C₁₋₄ hydrocarbyl or halogen;
A is a carbon- or heteroatom-containing linker group having a linking chain length of one or two atoms;
R³ is a monocyclic or bicyclic heteroaryl group containing from five to twelve ring members;
each group R⁴ is independently selected from hydrogen, hydroxy, halogen, nitro, cyano, a monocyclic heterocyclic group having up to seven ring members, a group N(R⁵)₂, a group C(O)N(R⁶)₂, a group SO₂N(R⁶)₂, a group R^{a}—R^{b} and a group Y; provided that no more than one group Y is present;
R^{a} is a bond, O, S, SO, SO₂, NH or N—C₁₋₄ hydrocarbyl;
R^{b} is C₁₋₈ hydrocarbyl optionally interrupted by O, S, SO, SO₂, NH or N—C₁₋₄ hydrocarbyl and optionally substituted by one or more substituents selected from hydroxy, amino, mono- or di-C₁₋₄ hydrocarbylamino, C₁₋₄ hydrocarbyloxy, oxo, C₁₋₄ hydrocarbylthio and halogen;
each group R⁵ is independently selected from hydrogen, C₁₋₄ alkyl, C₁₋₄ acyl and C₁₋₄ alkylsulphonyl;
each group R⁶ is independently selected from hydrogen and C₁₋₄ hydrocarbyl;
Y is a group —N(R₇)—C(O)—R⁸ or —N(R⁷)—SO₂—R⁸;
R⁷ is hydrogen, C₁₋₄ hydrocarbyl or a group C(O)—R⁸ or SO₂—R⁸;
R⁸ is selected from C₁₋₁₀ hydrocarbyl, C₁₋₁₀ hydrocarbylamino, C₁₋₁₀ hydrocarbylthio, C₁₋₁₀ hydrocarbyloxy, and aryl, arylamino, arylthio and aryloxy groups, the aryl moieties of which are carbocyclic or heterocyclic and have from five to twelve ring members, each substituent group R⁸ being optionally substituted by one or more groups R⁴ other than Y; or R⁷ and R⁸ together with the nitrogen and carbon or sulphur atoms to which they are attached are linked to form a ring structure of 4 to 7 ring members;
wherein R⁰ is other than a 2-(2,4-diamino-6-triazinyl)ethyl group when, in combination, U, T, V and W are all CH, and R¹ and R² are both hydrogen;
and provided that when the group -A-R³ contains an acidic substitituent group selected from carboxylic, phosphonic and sulphonic acids and tetrazoles, or contains a —C(O)NSO₂— group, or when -A- is —C(O)N— and the nitrogen atom of the group A is linked directly to a furan or thiophene ring, then either R¹ is -A-R³ and both R⁰ and R² are hydrogen, or R⁰ is -A-R³ and R¹ is hydrogen.

75. A compound of the formula (I): 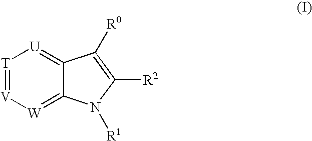
wherein U, T, V and W are each a nitrogen atom or a group CR⁴ provided that no more than three of U, T, V and W are nitrogen atoms;
R⁰ is hydrogen, C₁₋₄ hydrocarbyl, halogen or a group -A-R³;
R¹ is hydrogen, C₁₋₄ hydrocarbyl or a group -A-R³; provided that only one of R⁰ and R¹ is a group -A-R³;
R² is hydrogen, C₁₋₄ hydrocarbyl or halogen;
A is a carbon- or heteroatom-containing linker group having a linking chain length of one or two atoms;
R³ is a monocyclic or bicyclic heteroaryl group containing from five to twelve ring members;
each group R⁴ is independently selected from hydrogen, hydroxy, halogen, nitro, cyano, a monocyclic heterocyclic group having up to seven ring members, a group N(R⁵)₂, a group C(O)N(R⁶)₂, a group SO₂N(R⁶)₂, a group R^{a}—R^{b} and a group Y;
provided that the compound of the formula (I) contains one group R⁴ which is a group Y;
R^{a} is a bond, O, S, SO, SO₂, NH or N—C₁₋₄ hydrocarbyl;
R^{b} is C₁₋₈ hydrocarbyl optionally interrupted by O, S, SO, SO₂, NH or N—C₁₋₄ hydrocarbyl and optionally substituted by one or more substituents selected from hydroxy, amino, mono- or di-C₁₋₄ hydrocarbylamino, C₁₋₄ hydrocarbyloxy, oxo, C₁₋₄ hydrocarbylthio and halogen;
each group R⁵ is independently selected from hydrogen, C₁₋₄ alkyl, C₁₋₄ acyl and C₁₋₄ alkylsulphonyl;
each group R⁶ is independently selected from hydrogen and C₁₋₄ hydrocarbyl;
Y is a group —N(R⁷)—C(O)—R⁸ or —N(R⁷)—SO₂—R⁸;
R⁷ is hydrogen, C₁₋₄ hydrocarbyl or a group C(O)—R⁸ or SO₂—R⁸;
R⁸ is selected from C₁₋₁₀ hydrocarbyl, C₁₋₁₀ hydrocarbylamino, C₁₋₁₀ hydrocarbylthio, C₁₋₁₀ hydrocarbyloxy, and aryl, arylamino, arylthio and aryloxy groups, the aryl moieties of which are carbocyclic or heterocyclic and have from five to twelve ring members, each substituent group R⁸ being optionally substituted by one or more groups R⁴ other than Y; or R⁷ and R⁸ together with the nitrogen and carbon or sulphur atoms to which they are attached are linked to form a ring structure of 4 to 7 ring members;
wherein R⁰ is other than a 2-(2,4-diamino-6-triazinyl)ethyl group when, in combination, U, T, V and W are all CH, and R¹ and R² are both hydrogen;
and provided that when the group -A-R³ contains an acidic substitituent group selected from carboxylic, phosphonic and sulphonic acids and tetrazoles, or contains a —C(O)NSO₂— group, or when -A- is —C(O)N— and the nitrogen atom of the group A is linked directly to a furan or thiophene ring, then either R¹ is -A-R³ and both R⁰ and R² are hydrogen, or R⁰ is -A-R³ and R¹ is hydrogen; and excluding the compound wherein in combination R¹ and R² are hydrogen, U, V and W are all CH and T is a carbon atom bearing an unsubstituted benzamido group.

76. A compound according to claim 75 wherein the linker group A is CH₂CH₂.

77. A compound according to claim 75 wherein R³ is a monocyclic heteroaryl group having six ring members.

78. A compound according to claim 75 wherein R³ is a pyridyl group or a pyrimidinyl group.

79. A compound according to claim 75 wherein R⁰ is a group -A-R³.

80. A compound according to claim 79 wherein R¹ is selected from hydrogen and methyl.

81. A compound according to claim 75 wherein R¹ is a group -A-R³.

82. A compound according to claim 75 wherein R² is selected from hydrogen and methyl.

83. A compound according to claim 75 wherein each of U, T, V and W is a group CR⁴.

84. A compound according to claim 83 wherein R¹ is a group -A-R³, and T is CR⁴ wherein R⁴ is Y.

85. A compound according to claim 83 wherein R¹ is a group -A-R³, and V is CR⁴ wherein R⁴ is Y.

86. A compound according to claim 84 wherein Y is a group —N(R⁷)—C(O)—R⁸.

87. A compound according to claim 85 wherein Y is a group —N(R⁷)—C(O)—R⁸.

88. A compound selected from:
3-(2-(4-pyridyl)ethyl)-5-(3-trifluoromethoxybenzamido)indole;
3-(2-(4-pyridyl)ethyl)-5-(3-trifluoromethylbenzamido)indole;
3-(2-(4-pyridyl)ethyl)-5-(3-fluoro-5-(1-N-morpholino)benzamido)indole;
1-(2-(4-pyridyl)ethyl)-5-(3-fluoro-5-(1-N-morpholino)benzamido)indole;
5-(phenylcarbamoylamino)-3-(2-(4-pyridyl)ethyl)indole;
5-(3-tert-butyl-1-phenylpyrazol-5-ylcarbamoylamino)-3-(2-(4-pyridyl)ethyl)indole;
3-(2-(2-(2-hydroxyethylamino)-4-pyrimidinyl)ethyl)indole;
3-(2-(2-(3-hydroxy-2-methyl-prop-2-ylamino)-4-pyrimidinyl)ethyl)indole;
3-(2-(2-((S)-(−)-α-methylbenzylamino)-4-pyrimidinyl)ethyl)indole;
3-(2-(2-((S)-(+)-α-methylbenzylamino)-4-pyrimidinyl)ethyl)indole;
6-(3-fluoro-5-(4-morpholino)benzamido)-3-(2-(4-pyridyl)ethyl)indole; and
6-(3-fluoro-5-(4-morpholino)benzamido)-1-(2-(4-pyridyl)ethyl)indole.

89. A pharmaceutical composition comprising a compound according to claim 75 and a pharmaceutically acceptable carrier.
